(19) United States
(12) Patent Application Publication
(10) Pub. No.: US 2004/0241657 A1
(43) Pub. Date: Dec. 2, 2004

(54) Liver related disease compositions and methods

(75) Inventors: Nila Patil, Woodside, CA (US); David R. Cox, Belmont, CA (US); Coleen R. Hacker, San Carlos, CA (US); David Hinds, Mountain View, CA (US); David Kershenobich, Mexico (MX); Naiping Shen, Saratoga, CA (US)

Correspondence Address: WILSON SONSINI GOODRICH & ROSATI, 650 PAGE MILL ROAD, PALO ALTO, CA, 943041050

(73) Assignee: Perlegen Sciences, Inc., Mountain View, CA

(21) Appl. No.: 10/447,685

(22) Filed: May 28, 2003

(51) Int. Cl. C12Q 1/68; C07H 21/04; C07K 14/47; C12N 9/64

(52) U.S. Cl. 435/6; 435/69.1; 435/320.1; 435/325; 530/350; 536/23.5; 435/226

(57) ABSTRACT

Composition and methods for use in the therapeutic and preventative treatment, study, diagnosis and prognosis of liver related disease, inflammatory disease and related conditions are disclosed. Also provided are kits and reagents for prognosis and diagnosis of liver related disease, inflammatory disease and related conditions.

### BACKGROUND

Cirrhosis is a liver related disease that is a leading cause of death in the United States. Roughly 25,000 to 30,000 Americans die from cirrhosis each year. Cirrhosis results from the replacement of normal, healthy tissue by scar tissue which blocks the flow of blood through the liver and prevents normal liver functions. Cirrhosis can be caused by genetic and non-genetic factors. In the United States, excessive alcohol consumption and hepatitis C are the most common causes for cirrhosis.

Many people with cirrhosis and other liver related disease conditions experience no symptoms in the early stages of the disease. However, as scar tissue replaces healthy tissue, liver functions begin to fail and a person may experience fatigue, exhaustion, loss of appetite, nausea, weakness and loss of weight. As the disease progresses, complications may develop as a result of the loss of liver functions.

Complications from liver related disease include, for example, edema and ascites (water accumulation in the leg and abdomen, respectively); bruising and bleeding as a result of loss of blood clotting proteins that are produced in the liver; jaundice (yellowing of the skin and eyes) as a result of liver failure to absorb bilirubin; itching as a result of bile products deposited in the skin; gallstones as a result of failure of bile to reach the gallbladder; toxins in the blood or brain as a result of liver failure to detoxify the blood which may lead to coma or death; hypersensitivity to medication as a result of failure of the liver to remove drugs from the blood at normal rates which results in longer circulation of existing drugs and toxins; portal hypertension as a result of increased blood pressure in the portal vein that carries blood from the intestines and spleen to the liver; varices (enlarged blood vessels) as a result of accumulation of blood in vessels of the stomach and esophagus due to inefficient blood flow through portal veins. Since such vessels have thin walls, over-accumulation of blood under high pressure can cause these vessels to burst. If they do burst, the result is a serious bleeding problem in the upper stomach or esophagus that requires immediate medical attention. Problems in other organs include to fluid accumulation in the abdomen, kidney failure and general immune system dysfunction as a result of infections from bacteria normally present in the intestines. For many people, these symptoms may be the first signs of liver related disease.

A diagnosis of liver related disease may be based on any of the above symptoms. In addition, laboratory tests, medical history and a family history of liver disease may be used to diagnose liver related disease. In a physical examination, a clinician noticing that a liver feels harder or larger than usual can order blood tests to determine liver functions. Other means for diagnosing liver related disease include the use of CAT scans, ultrasound, radioisotope scanning and laparoscopy (a fiber optic system that allows for visualization of internal organs). Furthermore, a liver biopsy may be used. A biopsy involves taking a small sample of tissue from the liver being tested and examining the sample for scarring or other signs of liver related disease.

While liver damage from cirrhosis cannot previously be reversed, damage from other conditions associated with liver related disease, such as fatty liver, is currently treated. Treatment for liver related disease varies according to its cause and the complications that a person is experiencing. For example, liver related disease caused by excessive alcohol consumption is treated by abstaining from alcohol consumption. Treatment for hepatitis-related cirrhosis involves medications, such as interferon and corticosteroids used to treat different forms of hepatitis. Liver related disease caused by Wilson's disease, which results in copper build-up in various organs, is treated with medications to remove the copper. Treatment for ascites and edema may be a low-sodium diet or diuretics to remove fluids from the body. Antibiotics may be prescribed for various infections associated with liver related disease. A low-protein diet may be prescribed to reduce the release of toxins, and laxatives may be used to absorb toxins and remove them from the intestines. Over the counter medication may be used to treat itching. For portal hypertension, blood pressure medication, such as a beta-blocker, may be used. For varices bleeds, clotting agents or rubber-band ligation may be used to stop bleeding.

When complications cannot be controlled or when the liver becomes so damaged from scarring or otherwise that it completely fails to function, a liver transplant may be necessary. A liver transplant removes a non-functioning liver and replaces it with a healthy one. Liver transplants can be very expensive ranging $75,000 to $250,000. In addition, the waiting list for a liver is very long—roughly 17,000 individuals are waiting for a liver donation in the United States at any given time. The survival rate for liver transplant is about 80 to 90 percent.

The greatest risk factor for developing liver related disease is excessive alcohol consumption. More than half of all cirrhosis cases are attributed to excessive alcohol consumption. In the United States, it is estimated that roughly 14 to 20 million Americans are heavy drinkers, and of those, nearly two million people suffer from some form of liver related disease, in particular alcohol liver disease.

Alcohol liver disease is sometimes divided into three pathologically distinct liver related disease symptoms: fatty liver, alcoholic hepatitis and cirrhosis. Fatty liver is the accumulation of fat within hepatocytes, which are the most common type of liver cells. Fatty liver is a reversible condition such that if a patient stops drinking alcohol, the symptoms may disappear. However, if the patient does not stop drinking, fatty liver can lead to steatohepatitis or inflammation of the liver, which in turn can cause scarring of the liver or cirrhosis. Inflammation is a defensive physiological response caused by tissue damage or injury often characterized by redness, heat, swelling and pain. One purpose of inflammation is to prevent the spread of injury and mobilize the defense mechanisms of the immune system. Inflammation often leads to the generation of free radicals that can destroy disease-causing microorganisms, but it can also destroy healthy tissue. It is known that long-term alcohol consumption prolongs the inflammatory process, leading to excessive production of free radicals. Other liver related disease conditions may also lead to inflammation.

Excessive alcohol consumption can also cause acute and chronic alcoholic hepatitis. A patient who suffers from alcoholic hepatitis is usually a chronic drinker with a recent episode of exceptionally heavy drinking. Alcoholic hepatitis can range from a mild with abnormal laboratory tests being the only indication of disease, to severe liver dysfunction with complications such as jaundice, hepatic encephalopathy (neurological dysfunction), ascites, bleeding esophageal varices, abnormal blood clotting and coma. Histologically, alcoholic hepatitis can be characterized by expansion and degeneration of hepatocytes and inflammation with neutrophils or sometimes Mallory bodies (abnormal aggregations of cellular intermediate filament proteins).

It is useful to identify genetic factors that are associated with resistance and susceptibility to liver related disease. Such genetic factors can be utilized for the development of diagnostics, prognostics, preventative and therapeutic treatments, as well as research tools for studying liver related disease.

### BRIEF SUMMARY

Methods and compositions for diagnosing, prognosticating, preventing, treating and investigating liver related disease, in particular cirrhosis and alcohol liver disease, are provided. In addition, the methods and compositions herein may be utilized for the diagnosis, prognosis, prevention, treatment and study of inflammation and other associated diseases.

Furthermore, methods for collecting samples for identifying genetic regions correlated with susceptibility to liver disease are provided. Such methods include collecting genomic samples from a first group of large alcohol consumers with cirrhosis and a second group of large alcohol consumers without cirrhosis wherein both of said first and second groups comprise individuals not clinically diagnosed with any of jaundice, spider angiomas, palmar erythema, abdominal collateral circulation, ascitis, hepatic encephalopathy, esophageal varices, portal hypertension, hepatitis, or esophageal varices.

### DETAILED DESCRIPTION

Throughout this disclosure various patents, patent applications, and publications are referenced and unless otherwise indicated, are incorporated by reference in their entirety and for all purposes.

It is understood that the terminology used herein is for the purpose of describing particular embodiments only and is not intended to limit the scope of the present invention.

The term “a” or “an” as used herein may mean one or more.

The term “another” as used herein may mean at least a second or more.

The term “associated gene” refers to a gene encompassing (i.e. containing a reference or variant allele position) one of the variant regions identified in Table 1 plus a genomic region 10 kb upstream and 10 kb downstream of such gene(s), and all associated gene products (e.g., isoforms, splicing variants, and/or modifications, derivatives, etc.).

The term “associated gene pathway” refers to any gene upstream of an associated gene or any gene whose product interacts with, binds to, competes with, induces, enhances or inhibits, directly or indirectly, the expression or activity of an associated gene; or any gene downstream of an associated gene or whose gene product is induced, enhanced or inhibited by an associated gene, directly or indirectly.

The term “complementary” can mean partially complementary or completely complementary and generally refers to the natural hydrogen bonding between purines and pyrimidines base pairs. The term “partially complementary” refers to instances where only some of the base pairs are bonded. The term “completely complementary” refers to instances where all or nearly all of the base pairs are bonded.

The term “derivative” refers to chemical modification of a nucleic acid, a protein or mimetic thereof. Examples of chemical modifications of a nucleic acid include replacement of hydrogen by an alkyl, an acyl or an amino group. A nucleic acid derivative can encode a polypeptide which retains, changes, inhibits or enhances essential characteristics or functions of the polypeptide which the natural nucleic acid encodes. A polypeptide derivative is one that is modified by glycosylation, pegylation or other process and that retains, changes, inhibits or enhances at least one characteristic or function (e.g., immunological response) of the polypeptide from which it was derived.

The term “stringent conditions” refers to conditions for hybridization of complementary nucleic acid wherein the presence of a nucleic acid may be detected. Different stringency conditions may be utilized under different circumstances. Stringent conditions depend on, for example, length of the nucleic acids, temperature and buffers. Generally, stringent conditions are selected to be about 5° C. lower than the thermal melting point (Tm) of a specific sequence at a defined ionic strength and pH. The Tm is the temperature (under defined ionic strength, pH and nucleic acid concentration) at which 50% of the complementary nucleic acids hybridize to a target nucleic acid at equilibrium. As target nucleic acids are generally present in excess, at Tm, 50% of the complementary nucleic acids are occupied at equilibrium. Typically, stringent conditions include a salt concentration of at least about 0.01 to 1.0 M Na ion concentration (or other salts) at pH 7.0 to 8.3 and the temperature is at least about 30° C. for short probes (e.g., 10 to 50 nucleotides). Stringent conditions can also be achieved with the addition of destabilizing agents such as formamide. For example, conditions of 5×SSPE (750 mM NaCl, 50 mM NaPhosphate, 5 mM EDTA, pH 7.4) and a temperature of 25-30° C. are suitable for allele-specific nucleic acid hybridizations.

The terms “isolated” and “purified” refer to a material that is substantially or essentially removed from or concentrated in its natural environment. For example, an isolated nucleic acid is one that is separated from the nucleic acids that normally flank it or other nucleic acids in a sample. In another example, a polypeptide is purified if it is substantially removed from or concentrated in its natural environment.

The term “liver related disease” refers to one or more diseases, conditions or symptoms or susceptibility to diseases, conditions or symptoms that involve directly or indirectly, the liver, the biliary ducts, the hepatic ducts, the cystic ducts or the gallbladder including the following: acute liver failure, Alagille syndrome, alcohol liver disease, Alpha 1—antitrypsin deficiency, autoimmune hepatitis, biliary atresia, chronic hepatitis, cirrhosis, cholestatic liver disease, cystic disease of the liver, fatty liver, galactosemia, gallstones, Gilbert's syndrome, hemochromatosis, hepatitis A, hepatitis B, hepatitis C, liver cancer, neonatal hepatitis, non-alcoholic liver disease, non-alcoholic steatohepatitis, porphyria, primary biliary cirrhosis, primary sclerosing cholangitis, Reye's syndrome, sarcoidosis, steatohepatitis, tyrosinemia, type I glycogen storage disease, viral hepatitis, Wilson's disease. The term also includes inflammatory diseases, conditions and/or symptoms, including but not limited to arthritis, rheumatoid arthritis, allergic rhinitis (hay fever), asthma, cardiovascular disease, chronic obstructive pulmonary disease, inflammatory bowel disease, and multiple sclerosis. In some embodiments, cancer and cardiovascular diseases are excluded.

The term “liver related disease nucleic acid” or “associated genomic region” means a nucleic acid, or fragment, derivative, variant or complement thereof, associated with resistance or susceptibility to liver related disease including, for example, coding and non-coding regions of an associated gene, and/or genomic regions spanning 10 kb immediately upstream and 10 kb immediately downstream of an associated gene, and variants thereof. The term also includes nucleic acids similarly related to genes in an associated gene pathway.

The term “liver related disease polypeptide” refers to any peptide, polypeptide, or fragment, derivative or variant thereof, associated with resistance or susceptibility to liver related disease, including a peptide or polypeptide regulated or encoded, in whole or in part, by an associated gene or genomic regions of 10 kb immediately upstream and downstream of an associated gene, or fragment, variants, derivative, or modifications thereof. The term also includes such polypeptides up- or down-stream in an associated gene pathway.

The term “modulate” refers to a change such as in expression, or lifespan, such as an increase, decrease, enhancement or inhibition of expression or activity.

The term “nucleic acid,” refers to a deoxyribonucleotide, ribonucleotide and/or a mimetic thereof, whether singular or in polymers, naturally occurring or non-naturally occurring, double-stranded or single-stranded, translated (e.g., gene) or untranslated (e.g. regulatory region), or any fragments, derivatives or complements thereof. A nucleic acid includes analogs (e.g., phosphorothioates, phosphoramidates, methyl phosphonate, chiral-methyl phosphonates, 2-O-methyl ribonucleotides) or modified nucleic acids (e.g., modified backbone residues or linkages) or nucleic acids that are combined with carbohydrate, lipids, protein or other materials or peptide nucleic acids (PNAs). A nucleic acid can include one or more polymorphisms, variations or mutations. Examples of nucleic acids include oligonucleotides, nucleotides, polynucleotides, nucleic acid sequences, genomic sequences, antisense nucleic acids, probes, primers, genes, regulatory regions, introns, exons, open-reading frames, binding agents, target nucleic acids and allele specific nucleic acids.

The terms “polypeptide,” “peptide,” “oligopeptide” and “protein” are used interchangeably to refer to a polymer of amino acids, PNAs or mimetics, of no specific length and to all fragments, isoforms, variants, derivatives and modifications thereof. A polypeptide may be naturally and non-naturally occurring. The term isoform refers to different gene products resulting from altered initiation sites or altered promoters of the same gene. The term variant when used to describe a polypeptide refers to variations in amino acid sequences, whether or not such variations result in conservative or non-conservative substitutions. The term modification include tags, labels, post-translational modifications or other chemical or biological modifications. In preferred embodiment a polypeptide is purified.

The term “probes” or “primers” refers to nucleic acids, mimetics and PNAs that can hybridize, in whole or in part, in a base-specific manner to a complementary strand. In particular, the term “primer” refers to a single-stranded nucleic acid that acts as a point of initiation of template-directed DNA synthesis (e.g., PCR primers) and the term “probe” refers to a single-stranded nucleic acid that is used to identify the presence or absence of a complementary nucleic acid.

The term “specific hybridization” refers to the ability of a first nucleic acid to bind, duplex or hybridize to a second nucleic acid in a manner such that the second nucleic acid can be distinguished or identified when that second nucleic acid is present.

The term “specific binding” refers to the ability of a first molecule (e.g., an antibody) to bind or duplex to a second molecule (e.g., a polypeptide) in a manner such that the second molecule can be distinguished when the second molecule is present in a complex mixture (e.g., total cellular polypeptides).

The term “substrate” refers to any rigid or semi-rigid support to which molecules (e.g., nucleic acids, polypeptides, mimetics) may be bound. Examples of substrates include membranes, filters, chips, slides, wafers, fibers, magnetic, or nonmagnetic beads, gels, capillaries or other tubing, plates, polymers, and microparticles with a variety of surface forms including wells, trenches, pins, channels and pores.

The term “variant” refers to nucleic acids, polypeptides or mimetics that are naturally or non-naturally altered, having one or more additions, deletions, substitutions and/or polymorphisms in nucleic acids or amino acids respectively. Variants nucleic acids include nucleic acids encoding a polypeptide, but which due to the degeneracy of the genetic code are not found in nature. Variant polypeptides include polypeptides encoded by another locus in the human genome or other organism's genome that have substantial homology, in whole or in part, to the polypeptides herein. The term “synonymous variant” refers to an altered nucleic acid that results in an identical amino acid sequence. The term “non-synonymous variant” refers to an altered nucleic acid that results in a changed amino acid sequence. Non-synonymous variants may be conservative or non-conservative. A “conservative variant” refers to an altered amino acid sequence that is functionally similar. A “non-conserved variant” refers to an altered amino acid sequence that is functionally dissimilar.

The term “vector” refers to any construct or composition by which the expression, transfer or manipulation of a nucleic acid may be accomplished or facilitated. For example, the term vector can be a viral, particle, a viral nucleic acid, plasmid, or a liposome, but typically a viral nucleic acid or a plasmid with appropriate transcription/translation control signals. An expression vector is a vector that is designed to promote the expression of the nucleic acid inserts.

I. The Liver and Liver Related Disease

The liver is the largest gland and organ in the human body. The liver consists of two main lobes, which are made up of thousands of lobules. These lobules are connected to small ducts that ultimately form the hepatic duct. The hepatic duct transports bile produced by liver cells to the duodenum (the first part of the small intestine) and then to the gallbladder. Bile acts as a detergent and helps with digestion of fats.

In addition to creating bile, the liver has many other important functions. For example, the liver stores glycogen, vitamins and other substances. Regulation of vitamin A and the blood's nitrogen levels takes place in the liver. The liver synthesizes blood-clotting factors, regulates blood volume and temperature and destroys old red blood cells. In addition, specialized liver cells known as hepatic cells conduct many metabolic and secretory functions such as neutralizing potential toxic chemicals and transforming substances into nutrients that the body can use. More than 60% of the liver is composed of hepatic liver cells. These cells carry out more metabolic functions than any other cell in the body.

Because the liver plays such a major role in the circulation and composition of blood, liver related disease has far reaching consequences. Roughly 8 to 10 million Americans suffer regularly from liver related disease. The most common conditions of liver related disease include: hepatitis C, acute liver failure, alcohol liver disease, non-alcoholic liver diseases, cancer of the liver, cholestatic liver disorder, viral hepatitis, autoimmune hepatitis, hemochromatosis and other metabolic disorders and primary sclerosing cholangitis. Other forms of liver related disease include hepatitis A, hepatitis B.

Acute liver failure occurs when there is a massive loss of hepatocytes. A new terminology has recently been suggested to acknowledge the clinical finding that patients with a more rapid onset of hepatic failure are more likely to recover. This new classification provides for three subclasses of acute liver failure: hyperacute liver failure (encephalopathy within seven days of the onset of jaundice), acute liver disease (encephalopathy 8 to 28 days after the onset of jaundice) and subacute liver failure (encephalopathy occurs 5 to 12 weeks after the onset of jaundice). The sub-classification of acute liver failure is not internationally standardized.

Alcohol liver disease is the most common form of liver related disease. More than half of all liver related disease deaths are attributable to over-consumption of alcohol. There are three distinct pathological symptoms or clinical conditions associated with alcohol liver disease: fatty liver, alcoholic hepatitis and alcohol-induced cirrhosis. Fatty liver is a condition associated with a significant intake of alcohol, (or, in some cases, over consumption of some foods such as carbohydrates) even in individuals who are not alcoholics, and is a reversible condition. In fatty liver, large fat droplets accumulate in the liver, leading to enlargement of the liver. Alcoholic hepatitis is a condition where the liver has been severely damaged by the effects of alcohol. This condition can range from mild to life threatening—mild having no obvious phenotypic affect, and severe being characterized by dysfunction, jaundice, neurological dysfunction, liver failure, abnormal blood clotting or coma. Alcohol-induced cirrhosis is a condition of permanent and irreversible damage to the liver where fibrosis and scarring leads to obstruction of blood flow. This prevents the liver from performing its critical functions of purifying the blood and nutrients absorbed from the intestine. The eventual outcome of cirrhosis is often total liver failure and death.

Nonalcoholic liver disease and non-alcoholic steatohepatitis describes a set of liver related disease symptoms that closely resembles alcohol liver disease but occurs in individuals consuming little or no alcohol. As with alcohol liver disease the initial step in the evolution of nonalcoholic liver disease is almost certainly the deposition of excess fat within the liver. In patients who develop nonalcoholic liver disease the fat is associated with inflammation and scarring which in a few cases may progress ultimately to cirrhosis. Most patients are asymptomatic to nonalcoholic liver disease and may only exhibit mild non-specific symptoms such as fatigue or lower-back pain. Nonalcoholic liver disease diagnosis is usually made after the finding of abnormal liver blood tests performed during routine investigations.

Cancer of the liver occurs as a result of uncontrolled growth and cell division in the liver. Eventually the liver fails to function because a significant portion of it is replaced by cancerous tissue. The most common primary malignant tumor of the liver is hepatocellular carcinoma. Chronic carriers of hepatitis B virus, and in particular those with chronic hepatitis or cirrhosis, have substantially increased risk of developing hepatocellular carcinoma. Recent research also indicates that patients who have long standing chronic hepatitis C virus infections are at an increased risk for developing hepatocellular carcinoma.

Cholestatic liver disease is failure of normal excretion of bile into the duodenum. Such failure results in characteristic clinical, biochemical and histologic alterations that can be intrahepatic or extrahepatic, acute or chronic. The most common causes of chronic cholestatic symptom in adults are primary biliary cirrhosis and primary sclerosing cholangitis. Primary biliary cirrhosis is the progressive destruction of bile ducts in the liver. It is ten times more frequent in women than in men and is usually diagnosed in people 30 to 60 years of age. Many patients have no symptoms and are diagnosed through the appearance of an abnormality on routine liver blood tests. Primary sclerosing cholangitis is a disease in which the bile ducts inside and outside the liver become narrowed due to inflammation and scarring. It usually begins in the 30's, 40's or 50's and is commonly associated with fatigue, itching and jaundice.

Autoimmune hepatitis is a progressive inflammation of the liver associated with an abnormality of the body's immune system and related to the production of antibodies. Common symptoms include fatigue, abdominal discomfort, aching joints, itching, jaundice, enlarged liver, and spider angiomas (tumors) on the skin. However, autoimmune hepatitis is usually self-limiting nucleic acid treatment includes bed rest, abstention from alcohol and corticosteroids which help reduce the symptoms.

Hemochromatosis is a genetic condition that causes the body to absorb and store too much iron. While many individuals have no symptoms of this condition, injuries to the liver can slowly lead to cirrhosis if the illness is not treated.

Hepatitis A and B are also viral infections that affect the liver. Hepatitis A is the inflammation of the liver usually caused by eating food or drinking water that has been contaminated with human excrements containing the hepatitis A virus. Symptoms of hepatitis A are similar to the flu. Hepatitis B is one of the most serious forms of hepatitis and is more common and more infectious than AIDS. Chronic hepatitis B may lead to scarring and cancer of the liver. Hepatitis B can be spread by contact with blood from an infected person (e.g., in cases of blood transfusion, needle sharing, acupuncture, tattooing, sexual intercourse, child birth) and possibly other bodily fluids (e.g., saliva).

Other forms of liver related disease include: Alagille syndrome, alpha 1-antitrypsin deficiency, biliary atresia, chronic hepatitis, cirrhosis, cystic disease of the liver, fatty liver, galactosemia, gallstones, Gilbert's syndrome, neonatal hepatitis, porphyria, Reye's syndrome, sarcoidosis, steatohepatitis, tyrosinemia, type I glycogen storage disease and Wilson's Disease. As inflammation often plays a key role in liver related disease, the term liver related disease also includes inflammatory diseases and conditions such as rheumatoid arthritis, allergic rhinitis (hay fever), asthma, cardiovascular disease, chronic obstructive pulmonary disease, inflammatory bowel disease and multiple sclerosis.

II. Liver Related Disease Nucleic Acids

As only 10 to 35 percent of heavy drinkers develop alcoholic hepatitis and only 20 percent of heavy drinkers develop cirrhosis, it is suggested that specific genetic factors may influence an individual's susceptibility and resistance to liver related disease. Other liver related disease conditions that are influenced by genetic factors include autoimmune hepatitis (a condition in which a patient's immune cells attack the liver); primary biliary cirrhosis (an autoimmune disease in which the bile tubes that drain bile from the liver are attacked); primary sclerosing cholangitis (a disease in which the bile tubes become blocked); alpha-1 antitrypsin deficiency (this enzyme protects the lung from destruction) and Wilson's disease (too much copper in the liver).

An association study reveals, by comparing case groups and control groups, that novel polymorphisms identified in Table 1 and surrounding genomic regions are correlated with susceptibility and/or resistance to liver related disease, in particular cirrhosis. In general, the case group was composed of individuals that develop cirrhosis after heavy alcohol consumption. The control group was composed of individuals that did not develop cirrhosis after heavy alcohol consumption. It is believed that individuals who are resistant or susceptible to liver related disease, or cirrhosis, over or under express liver related disease polypeptides and/or liver related disease nucleic acids.

Table 1 below identifies variants correlated with resistance or susceptibility to liver related disease. In particular, Table 1, column 1 identifies SNP identification number for each variant. Table 1 column 2 identifies the locus in which each variant is located, if such locus in known. Table 1, column 3 identifies the chromosomal location or position for each variant according to Build 33 of the human genome.

Table 1, column 4 identifies the relative allelic frequency for each variant as calculated according to the methods in U.S. application Ser. No. Unassigned, entitled “Apparatus and Methods For Analyzing And Characterizing Nucleic Acid Sequences,” (Attorney Docket No. 29202-702), filed on Apr. 3, 2003, assigned to the same assignee as the present application. Briefly, the difference in relative allelic frequency (delta.p.hat) is equal to the relative reference allelic frequency in a case group minus the relative reference allelic frequency in a control group. The relative reference allelic frequency can be calculated according to the following equation:

P′=I_{reference}/Iₐₗₜₑᵣₙₐₜₑ+I_{reference}.

A positive difference in relative allelic frequency indicates that the reference variant is associated with the case group, or susceptibility to liver related disease. A negative difference of relative allelic frequency indicates that the alternate variant is associated with control group or resistance to liver related disease.

Table 1, column 5 identifies the reference and alternate variant (novel) nucleotide bases associated with resistance or susceptibility to liver related disease. The reference variants are identified in Build 33 of the human genome. The alternate variants are novel mutations or polymorphisms in the human genome identified by association studies and genotyping analyses. Bold variants are associated with the resistance to liver related disease. Underlined variants are associated with susceptibility to liver related disease. Additional variants can be used in addition to those identified in Table 1, including those in haplotype blocks with the variants identified in Table 1, which can be identified according to in U.S. Ser. No. 10/106,097 entitled “Methods For Genomic Analysis”, filed Mar. 26, 2002, assigned to the same assignee as the present application. Variants in a haplotype block with a variant associated with resistance to liver related disease are also associated with resistance to liver related disease; similarly variants in a common haplotype block with a variant associated with susceptibility to liver related disease identified in Table 1 are also associated with susceptibility to liver related disease.

Table 1, column 6 identifies the 12 nucleotide bases upstream and 12 nucleotide bases downstream of each reference variant

TABLE 1

| | | Chromosome/ | Delta | Ref/ | |
|---|---|---|---|---|---|
| SNP ID | Locus | Position | Phat | Variant | Context −12 to +12 |
| 2058070 | TRHDE | Chromosome: | −0.0802 | A/G | GGACTCTTACTATTACTGAATTCT |
| | | 12/72677975 | | | |
| 3607795 | AVEN | Chromosome: | 0.0914 | A/G | TCCTCAAATACAATGAAGTGCCCAC |
| | | 15/31737404 | | | |
| 1385057 | | Chromosome: | −0.0987 | T/C | CCAAAAAGCCCATGTATGTGCTGTC |
| | | 2/180740253 | | | |
| 1836665 | | Chromosome: | −0.0634 | T/C | GACATACAGTGTTATCAGTTGTAAT |
| | | 18/65319504 | | | |
| 3196364 | | Chromosome: | −0.1212 | A/G | TTTTTCTCTGATAACTGTTGAAAGA |
| | | 16/79942295 | | | |
| 3195888 | | Chromosome: | 0.0843 | C/G | TTGTAAATTGCTCTATAAACACATC |
| | | 16/79794941 | | | |
| 3416625 | | Chromosome: | −0.0745 | T/A | TGGCCTCAACCTTACAAAGATATGG |
| | | 4/112175641 | | | |
| 2998526 | | Chromosome: | −0.0669 | C/A | ATGTTCACATTACTCAATCTGAAAC |
| | | 4/161525279 | | | |
| 3014180 | DRD3 | Chromosome: | 0.0676 | G/A | TCATCACTGTACGCCTAAATTCTAC |
| | | 3/115123242 | | | |
| 1898629 | | Chromosome: | −0.0664 | A/G | GGTCGAAACCAAAGTCTGGTGTTAA |
| | | 8/34165683 | | | |
| 1396180 | STAT1 | Chromosome: | 0.0618 | G/A | ATATGCTGGACCGTCAGGCAATGGG |
| | | 2/191840092 | | | |
| 1873257 | | Chromosome: | −0.075 | A/G | CTGACTTCCAAGATATCACAGATAA |
| | | 8/16343592 | | | |

SNP 2058070 is located on chromosome 12 at position 72677975 in locus TRHDE. The TRHDE gene, also known as “Thyrotropin-Releasing Hormone-Degrading Ectoenzyme” gene, encodes an enzyme associated with plasma membrane that catalyzes in extracellular space. See Schomburg L., (1999) Eur. J. Biochem. 265, 415-422. TRHDE is a zinc-dependent metallopeptidase that inactivates Thyrotropin-Releasing Hormone (TRH) in a highly specific manner by cleaving a L-pyroglutamyl/histindinyl bond at the amino terminal end. TRHDE's specificity for TRH is very unusual and the ectoenzyme may be a highly specific terminator of TRH.

TRH is an important extracellular multi-functional signaling neuropeptide that stimulates the secretion of hormones, which play important roles in neuromodulation and neurotransmission in the central and peripheral nervous systems. TRH stimulates the release of Thyrotropin or Thyroid Stimulating Hormone (TSH) from the thyroid gland, which causes thyroid cells to convert intracellular thyroglobulin to thyroxine (T4) and triiodothyronine (T3), both of which are released into the bloodstream; T4 as the storage form and T3 as the more active form of the thyroid hormone. T4 can be converted into triiodothyronine (T3) in the thyroid, brain, liver, bloodstream and various other tissues, with a measurable byproduct known as reverse T3 (rT3). T4 increases the levels of enzymes that are responsible for metabolic reactions, including those in the liver and in the mitochondria. T3 directly affects energy metabolism in mitochondria, facilitating rapid protein synthesis and gene transcription. Increases in protein degradation, fatty acid production and oxygen consumption occur as a result of these activities. Serum T3 and T4 are involved in feedback on the production of TSH to maintain appropriate concentrations of T3 and T4.

Deficiencies in TSH levels cause a reduction in the quantity of serum T3 and T4. This can lead to a condition called hypothyroidism where thyroid function is gradually lost. Hypothyroidism typically requires synthetic hormone replacement therapy and can increase the risk of developing coronary artery disease and in some cases result in death.

T3's influence on metabolism results in higher amounts of protein degradation thereby increases the levels of free fatty acids and overall oxygen usage. These metabolic processes create a state of oxidative stress that can damage tissue because reactive oxygen species or free radicals are endogenously produced. T3 affects cellular responses to oxidative stress, including one involving Tumor Necrosis Factor (TNF), which is known to activate and increase replication of genes that respond to free radical attack. A study in rat liver Kuppfer cells reveals that an increase in the amount of T3 causes a significant elevation in the amount of circulating TNF-α. See Fernandez V., (2002) Free Radical Research, July;36(7):719-25. Thyroid dysfunction caused by abnormal levels of T3 and T4 has been noted in individuals with liver related disease conditions. T3 has been found to increase metabolism in the mitochondria and trigger rapid protein synthesis. Cirrhosis patients have significantly lower concentrations of serum T4, which causes a decrease in the amount of thyroid-binding globulin available, and low levels of serum T3. In contrast the concentration of rT3 is significantly higher in liver. Deficient amounts of serum T3 have also been noted in individuals with chronic liver disease who develop acute hepatitis.

Alcohol is known to increase the rates of oxygen metabolism and free radical production in the liver, thereby elevating the risk of injury to the liver through oxidative stress. A study in rat liver microsomes shows that the levels of oxygen consumption and free radical production were significantly reduced by the application of an antithryoid drug, propylthiouracil (PTU), in rats receiving ethanol chronically. See Ross A.D., (1995) Biochemical Pharmacology, Mar 30;49(7):979-89. The small molecule PTU inhibits the conversion of T4 into T3. The presence of alcohol also impedes T3 degradation in the brain. Elevated levels of T3 in the brain act as an anti-depressant.

SNP 3607795 is located on chromosome 15 at position 31737404 in the AVEN locus. The AVEN gene encodes a 362-amino acid protein that is conserved in numerous mammalian species and is an inhibitor of caspase activation. AVEN inhibits caspase activation by binding to BCLXL and the caspase regulator APAF1. BCLXL is an antiapoptotic BCL2 family member that functions at multiple stages in the cell death pathway. A Northern blot analysis of AVEN detects a 1.7-kb AVEN transcript in all adult tissues with highest expression in heart, skeletal muscle, kidney, liver, pancreas and testis. Other cell lines also expressed AVEN. However, only mutants of BCLXL that retain their antiapoptotic activity are capable of binding AVEN. Furthermore, AVEN interferes with the ability of APAF1 to self-associate, suggesting that AVEN impairs APAF1-mediated activation of caspases. This is consistent with the fact that AVEN inhibits the proteolytic activation of caspases in a cell-free extract and suppresses apoptosis induced by APAF1 plus caspase-9. Thus, AVEN may represent a novel class of cell death regulators. See Molec. Cell. 6:31-40 (2000).

SNP 1385057 is located on chromosome 2 at position 180740253 in locus 344086, upstream of KIAA1604.

SNP 1836665 is located on chromosome 18 at position 65319504 in locus 342798. Locus 342798 is immediate downstream of locus 342797 (18q22.1 similar to SECIS-binding protein 2). It may have a role in regulating SBP2 protein. SBP2 is essential for the function of selenoproteins, which provide defense against oxidant molecules. SBP2 is SECIS (selenocysteine insertion sequence) binding protein 2. Studies in rat have shown that SBP2 is essential for the cotranslational insertion of sec into selenoproteins. Thus, the binding activity of SBP2 may be involved in preventing termination at the UGA/sec codon. Selenoprotein has several important biological functions. For example, selenium (Se) is an essential metal that is required for normal antioxidant metabolism, reproduction and thyroid function. Compromised thyroid activity has been correlated with liver disease in the case of TRHDE, TRH, TSH, PRL, thyroid hormones T4 and T3.

Selenoprotein is an important coenzyme for the glutathione peroxidase detoxification system. Because of this, selenium neutralized peroxides that proliferate under oxidate stress and consequently protects cell membranes from free radical damage. (which occurs in alcohol liver disease as well). Selenium often combines with amino acids and forms selenoproteins. Also, viruses might benefit from being directly involved in this selenoprotein encoding process by monitoring selenium levels in the cell. Consequently, this viral behavior could act as a barometer for increasing or decreasing viral reproduction. If cellular selenoprotein levels fall, the virus might become more active and produce more viruses that attack new cells. If selenoprotein levels rise, the virus may remain in a dormant state for longer periods of time or remain permanently dormant. Research papers have reported that RNA viruses, including hepatitis C virus, encode selenium-dependent glutathione peroxidase genes. In view of this concept, it is entirely possible that a specific viral gene could generate a selenium shortage in the host. And in this way, a selenium deficiency could stimulate viral proliferation and thus promote the progression of hepatitis C. To continue, in that case, the addition of selenium might act as a “birth control pill” for the virus, and thus show down it's reproduction mechanisms. According to several investigators this could give the immune system a chance to control the hepatitis C or HIV disease process.

SNP 3196364 is located on chromosome 16 at position 79942295 in an unknown locus, upstream of locus 342482.

SNP 3195888 is located on chromosome 16 at position 79794941 in an unknown locus.

SNP 3416625 is located on chromosome 4 at position 112175641 in an unknown locus.

SNP 2998526 is located on chromosome 4 at position 161525279 in locus 351666. Locus 351666 contains domains that can be found in NADH:ubiquinone oxidoreductase subunit 5 (chain L)/Multisubunit Na+/H+ antiporter, MnhA subunit, and may be involved in energy production and conversion, inorganic ion transport and metabolism.

SNP 3014180 is located on chromosome 3 at position 115123242 in the DRD3 locus. The DRD3 gene, also known as “Dopamine Receptor D3” gene, encodes a D3 subtype dopamine receptor that is involved in neuronal development and signal transduction. The D3 subtype receptor inhibits adenylyl cyclase through inhibitory G-proteins. D3 receptor differs from D1 and D2 receptors in its pharmacology and signaling system as it represents both an autoreceptor and a postsynaptic receptor. Moreover, the D3 receptor is expressed in phylogenetically older regions of the brain, such as the limbic areas. These areas are associated with cognitive, emotional and endocrine functions, suggesting that DR3 receptor plays a role in cognitive and emotional functions. DR3 receptor is a target for drugs treating schizophrenia and Parkinson diseases. Alternative splicing of DR3 results in five transcript variants encoding different isoforms: a, b, c, d and e.

SNP 1898629 is located on chromosome 8 at position 34165683 in locus 346828, downstream of locus 137107. Locus 137107 contains domains found in ribosomal protein L1 and ribosomal protein L1p/L10e family, which are involved in translation, ribosomal structure and biogenesis. The L1p/L10e family includes prokaryotic L1 and eukaryotic L10. Thus, SNP 1898629 may be involved in translation, ribosomal structure and biogenesis through regulation and interaction with locus 137107.

SNP 1396180 is located on chromosome 2 at position 191840092 in the STAT1 locus. The STAT1 gene, also known as the “Signal Transducer And Activator of Transcription 1” gene, is a member of the STAT family and is involved in apoptosis in response to stresses, such as heat or ischemia. STATs are a family of cytoplasmic transcription factors known to play a major role downstream of the signal transduction pathways related to immunological responses and are part of the JAK/STAT intracellular signal transduction pathway. Following the extracellular binding of growth factors and cytokines to their cell surface receptors, receptor subunits oligomerize to form activated transmembrane receptor complexes. The intracellular receptor chain serves as a substrate for the JAK family of protein tyrosine kinases. Four members have been characterized in humans: JAK1, JAK2, Tyk2 and JAK3. JAKs are activated after ligand binding and the formation of a receptor complex. JAK activation eventually leads to the tyrosine phosphorylation of receptor chains, which creates binding sites for STATs. The STAT family consists of 6 transcription factors (STAT1 through STAT6) which have seven common homology domains, five of which are known to be necessary for STAT function. The homology domains include a conserved 140 amino acid amino-terminal region required for activity, a heptad leucine repeat region approximately 200 amino acids from the amino-terminal end in STAT1 required for activity, an SH2 domain involved in phospho-tyrosine binding, a carboxy-terminal tyrosine residue that is phosphorylated to activate STATs and a carboxy-terminal serine phosphorylation site in STAT1 that is involved in maximal activation of STAT1. A DNA binding domain is located approximately 400 amino acids from the amino-terminus of each STAT.

The STAT1 gene encodes two alternatively spliced RNA products that have been termed STAT1α (91 kDa) and STAT1β (86 kDa). These two forms are identical up to a conserved tyrosine residue at position 701 located approximately 40 amino acids from the carboxyl-terminus. Following cell surface binding of the polypeptide hormone, interferon γ(IFN-γ), a receptor complex is formed and tyrosine residues on both the intracellular receptor chains and on JAK1 and JAK2 are phosphorylated. Then, STAT1 is recruited to the cell membrane, binds phosphotyrosine residues on the receptor chain through its SH2 domain and is subsequently activated by phosphorylation on its tyrosine 701 and serine 727 residues. A study reveals that excessive amounts of tyrosine-phosphorylated STAT1 can induce an inflammatory cytokine response in multiple sclerosis patients. See Feng X., (2002) Journal of Neuroimmunology Aug;129(1-2):205-15. Under normal circumstances, activated STAT1 is translocated to the cell nucleus where it interacts with STAT DNA binding elements upstream of target genes. STAT1 initiates transcription of genes that are involved in the anti-viral and anti-tumor actions of interferons. Ligand binding to cell surface receptors by INFγ and a multifunctional proinflammatory cytokine, tumor necrosis factor-alpha (TNFα), induces programmed cell death or apoptosis. STAT1 is involved in the activation of cellular cysteine endopeptidases or caspases, which are involved in the process of apoptosis. See Kumar A., (1997) Science, Nov 28;278(5343):1630-2. Further STAT1 characterization study shows that cells lacking STAT1 have a reduced ability to undergo apoptosis in response to stress in the form of heat and a low oxygen state (ischemia). Janjua, S., (2002) Cell Death & Differentiation, Oct;9(10):1140-6. In addition the study reveals that the STAT1 carboxyl-terminal activation domain, including tyrosine 701 and serine 727, is necessary and required for stress-induced cell death. Moreover, the isolated carboxyl-terminal domain of STAT1 is able to enhance stress-induced apoptosis on its own, absent the other STAT1 functional regions, including the DNA binding domain. Negative regulation of the JAK/STAT pathway occurs through a couple of mechanisms. A family of suppressor proteins known as suppressor of cytokine signaling (SOCS) are involved in negative regulation of the JAK/STAT pathway by binding JAKs to inhibit their activity. Three SOCS have been identified (SOCS-1, SOCS-2 and SOCS-3). Down regulation of JAK/STAT activity also occurs through the de-phosphorylation of receptor complexes or JAKs by a family of phosphatases. The members include SHP-1, SHP-2, CD45 and PTP-1 B. A nuclear STAT1 protein tyrosine phosphatase TC45 has been described in an identification study. See ten Hoeve, J. (2002) Molecular and Cellular Biology, August;22(16):5662-8. TC45 de-phosphorylates STAT1 in the nucleus to inactivate it. Individuals with viral hepatitis and other liver related diseases are known to have high serum levels of TNFα, which inhibit signaling of another cytokine, Interferon α (IFNα) in the liver. Patients with Hepatitis C virus (HCV) are subjected to liver damage and application of IFNα is one treatment for people with chronic HCV. A study addresses the mechanism by which TNFα is able to inhibit IFNα signaling in vivo in mouse liver. See Hong F., (2001) FASEB Journal, July;15(9):1595-7. The experiments show that administration of INFα stimulates STAT1 activation but injection of TNFα suppresses tyrosine phosphorylation of INFα-activated STAT1.

STAT1 is upstream of STAT4, another member of the STAT family, which is essential for IL12 signal transduction and T helper cell differentiation. However, STAT1 is expressed ubiquitously, whereas STAT4 is expressed in specific tissues including spleen, heart, brain, peripheral blood cells, and testis.

The mouse STAT4 gene encodes a 779 amino acid protein (89 kDa), which is activated directly by the interferons, IFNα and IFNβ. STAT4 is known to mediate the signaling of cytokines that regulate the proliferation, differentiation and functional capacity of lymphocytes, also know as interleukins. STAT4 knock-out mice have defective lymphocytes that no longer proliferate in response to interleukin 12 (IL-12), fail to produce IFNγ and are unable to express natural killer cell cytotoxicity.

The human STAT6 gene encodes an 848 amino acid protein (94 kDa). STAT6 is involved in mediation of interleukin-lymphocyte signaling. STAT6 is known to mediate interleukin 4 (IL-4) signaling, T helper cell differentiation, expression of cell surface markers, class switching of immunoglobulins. An expression study reveals that and the induction of Bcl-2L/Bcl-xc1 expression. B-Lymphocytes in STAT6 knock-out mice fail to proliferate and do not mature in response to interleukin 4 (IL-4). In addition the T-lymphocytes in such animals exhibit an impaired ability to differentiate and proliferate.

STAT6 is also involved in the direct activation of genes in the Bcl-2 family, which includes both apoptosis-promoting genes (e.g., bax, bik, bad, bid, bcl-xs) and apoptosis-inhibiting genes (e.g., bcl-2, mcl-1, and bcl-xL, bcl-w). In any living cell, the relative ratio of anti- and pro-apoptotic Bcl-2-family proteins (and other proteins) dictates the ultimate fate of the cell (whether the cell becomes apoptotic leading to cell death, or malignant leading to uncontrolled growth. IL-4 is known to be an effective inhibitor of apoptosis and following activation STAT6 is capable of being transported to the nucleus to initiate IL-4 responsive genes. A biochemical study reveals that STAT6 is a critical factor in protecting primary B lyrnphocytes from apoptosis. See Wurster A. L., (2002) Journal of Biological Chemistry, July 26;277(30):27169-75. The experiments show that STAT6 directly activates transcription of the anti-apoptotic factor, Bcl-xL.

Furthermore, the STAT family of transcription factors is also involved in the expression of the hormone TRH. T3 normally regulates the expression of TRH at the transcriptional level. A series of experiments in the hypothalamic signaling pathways reveals that a starvation-induced reduction in the level of TRH in rodents can be reversed through the application of the protein leptin, a 16 kDa protein that plays a critical role in regulating body weight by inhibiting food intake and stimulating energy expenditure. See Harris M., (2001) Journal of Clinical Investigation, January, 107(1):111-20. Leptin upregulates TRH expression through two separate pathways. First its signaling pathway directly affects TRH gene expression through phosphorylation of STAT-3. Phosphorylated STAT-3 activates transcription of TRH by binding to a STAT-response element in the TRH promoter. Leptin indirectly affects TRH expression through its activation of the POMC gene, which is responsible for the production of α-melanocyte stimulating hormone (α-MSH), a candidate hormone for the regulation of TRH expression. α-MSH is a ligand for the melanocortin-4 receptor (MCR4), which plays an important role in regulating appetite and body weight. Activation of the melanocortin signaling system leads to the phosphorylation of cAMP response element binding protein (CREB), which binds to a CREB-response element in the TRH promoter, thereby increasing transcription of TRH. In mice, leptin has only been shown to activate STAT3 and no other STAT family member. In addition leptin only induces STAT activation in the hypothalamus of the mouse, but no other tissue.

Chronic ethanol consumption is known to increase body fat and circulating leptin levels. A study of the effect of alcohol intake on the expression of leptin receptors and STAT signaling molecules reveals that STAT levels are affected by alcohol. See Obradovic T., (2002) Alcohol Clinical Experimental Research, February;26(2):255-62. The study shows that STAT3 expression in the mouse hypothalamus was reduced by ethanol consumption but STAT1 expression was significantly elevated in the perigondal fat of ethanol-consuming mice.

In response to cytokines and growth factors, STAT family members are phosphorylated by receptor associated kinases to form homo- or heterodimers that translocate to the cell nucleus where the STAT family members act as transcription activators. STAT1 can be activated by various ligands including interferon-alpha, interferon-gamma, EGF, PDGF and IL6. STAT1 mediates the expression of a variety of genes, which is thought to be important for cell viability in response to different cell stimuli and pathogens.

Cells lacking STAT1 show reduced apoptosis in response to heat or ischaemia. Expression of STAT1 in these cells does not enhance cell death but restores sensitivity to stress-induced death. Data also suggests that down-regulation of interferon (IFN)-gamma-medicated nuclear STAT1 binding in hepatocytes involves both dephosphorylation by mitogen-activated protein kinase phosphatase 1 (MKP-1) and degradation via proteolysis by the ubiquitin-dependent proteasome pathway.

SNP 1873257 is located on chromosome 8 at position 16343592 in an unknown locus.

The variants and associated genomic regions identified in Table 1 can be used to identify, isolate and amplify nucleic acids associated with resistance or susceptibility to liver related disease. Such nucleic acids can be used for prognosis, diagnosis, prevention, treatment and further study of liver related disease.

In one embodiment, nucleic acids disclosed herein that can specifically hybridize to an associated genomic region, are identified in Table 1. Nucleic acids provided for herein can, in some embodiments, specifically hybridize to a genomic sequence having one or more variants identified in Table 1, column 5, and/or other variants in common haplotype blocks as the variants in Table 1, column 5. Methods for identifying variants in a common haplotype block are provided in U.S. Ser. No. 10/106,097 entitled “Methods For Genomic Analysis,” filed Mar. 26, 2002, assigned to the same assignee as the present application.

In a preferred embodiment, the nucleic acids herein are associated with resistance or susceptibility to liver related disease. For example, a nucleic acid associated with resistance to liver related disease is one that can specifically hybridize to a genomic region having one or more variants identified in Table 1, column 5 in bold and/or one or more variants in common haplotype blocks with the variants in bold. A nucleic acid associated with susceptibility to liver related disease is one that is differentially expressed in individuals having a phenotype of susceptibility to liver related disease or a nucleic acid having one or more variants associated with susceptibility to liver related disease. For example, a nucleic acid associated with susceptibility to liver related disease is one that can specifically hybridize to a genomic region having one or more variants identified in Table 1, column 5 in underline or one or more variants in common haplotype block with the variants in underline.

In more preferred embodiments, a set of nucleic acids is provided that can specifically hybridize to at least 2 variants, preferably at least 3 variants, at least 4 variants, at least 5 variants, at least 6 variants, at least 7 variants, at least 8 variants, or at least 9 variants associated with resistance to liver related disease such as those identified in Table 1 column 5 in bold, and/or variants in common haplotype blocks as the bold variants. Similarly, a set of nucleic acids may be provided that can specifically hybridize to at least 2 variants, preferably at least 3 variants, at least 4 variants, at least 5 variants, at least 6 variants, at least 7 variants, at least 8 variants, or at least 9 variants associated with susceptibility to liver related disease such as those identified in Table 1, column 5 in underline, and/or variants in common haplotype blocks as the underlined variants.

A nucleic acid can be single stranded or double stranded. It can also be coding (e.g., exon) or non-coding sequence (e.g., introns, exon outside coding region, and 3′ or 5′ untranslated regions) or a combination of coding and non-coding nucleic acids. In a preferred embodiment, a coding liver related disease nucleic acid is one that can specifically hybridize to the complete coding region of an associated genomic region, or to one ore more exons of an associated genomic region or to one or more open reading frames of an associated genomic region.

A nucleic acid provided herein can be fused to another molecule, such as a tag sequence, a reporter gene or a fusion protein. A sequence tag encodes a polypeptide that can assist in isolation or purification of the protein product (e.g., glutathione-S-transferase (GST) fusion protein or a hemagglutinin A (HA) polypeptide). A reporter gene also encodes an easily assayed protein and is often used to replace other coding regions whose protein products are difficult to assay. A fusion protein is formed by the expression of a hybrid nucleic acid made by combining two nucleic acid sequences.

Conditions for nucleic acid hybridization vary depending on the buffers used, length of nucleic acids, ionic strength, temperature, etc. The term “stringency conditions” for hybridization refers to the incubation and wash conditions (e.g., conditions of temperature and buffer concentration) that permit hybridization of a first nucleic acid to a second nucleic acid. The first nucleic acid may be perfectly (e.g. 100%) complementary to the second or may share some degree of complementarity, which is less than perfect (e.g., more than 70%, 75%, 85%, or 95%). For example, certain high stringency conditions can be used which distinguish perfectly complementary nucleic acids from those less complementary. High stringency, moderate stringency and low stringency conditions for nucleic acid hybridization are known in the art. Ausubel, F. M. et al., “Current Protocols in Molecular Biology” (John Wiley & Sons 1998), pages 2.10.1-2.10.16; 6.3.1-6.3.6. The exact conditions which determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, equivalent conditions can be determined by varying one or more of these parameters while maintaining a similar degree of identity or similarity between the two nucleic acid molecules. Typically, conditions are used such that sequences at least about 60%, at least about 70%, at least about 80%, at least about 90% or at least about 95% or more identical to each other remain hybridized to one another. By varying hybridization conditions from a level of stringency at which no hybridization occurs to a level at which hybridization is first observed, conditions which will allow a given sequence to hybridize (e.g., selectively) with the most similar sequences in the sample can be determined. Exemplary conditions are described in Krause, et al., Methods in Enzymology, (1991) 200:546-556 and in Ausubel, et al., “Current Protocols in Molecular Biology”, (John Wiley & Sons 1998), which describes the determination of washing conditions for moderate or low stringency conditions. Washing is the step in which conditions are usually set so as to determine a minimum level of complementarity of the hybrids. Generally, starting from the lowest temperature at which only homologous hybridization occurs, each ° C. by which the final wash temperature is reduced (holding SSC concentration constant) allows an increase by 1% in the maximum extent of mismatching among the sequences that hybridize. Generally, doubling the concentration of SSC results in an increase in TM of ˜17° C. Using these guidelines, the washing temperature can be determined empirically for high, moderate or low stringency, depending on the level of mismatch sought. For example, a low stringency wash can comprise washing in a solution containing 0.2×SSC/0.1% SDS for 10 min at room temperature; a moderate stringency wash can comprise washing in a prewarmed solution (42° C.) solution containing 0.2×SSC/0.1% SDS for 15 min at 42° C.; and a high stringency wash can comprise washing in prewarmed (68° C.) solution containing 0.1×SSC/0.1% SDS for 15 min at 68° C. Furthermore, washes can be performed repeatedly or sequentially to obtain a desired result as known in the art. Equivalent conditions can be determined by varying one or more of the parameters given as an example, as known in the art, while maintaining a similar degree of identity or similarity between the target nucleic acid and the primer or probe used.

In preferred embodiments, the nucleic acids herein are perfectly complementary to identified genomic regions. Furthermore, a nucleic acid is preferably isolated. For example, a genomic DNA nucleic acid is isolated when it is separated from the chromosome with which the genomic DNA is naturally associated and/or amplified. Nucleic acids can be isolated and amplified using polymerase chain reaction (PCR) techniques known in the art. See Erlich, H. A., “PCR Technology: Principles and Applications for DNA Amplification” (ed. Freeman Press, NY, N.Y., 1992); Innis M. A., et al., “PCR Protocols: A Guide to Methods and Applications” (Eds. Academic Press, San Diego, Calif., 1990).

1. Probes and Primers

The nucleic acids herein can be used as probes and primers in various assays. The terms “probe” and “primer” refer to nucleic acids that hybridize, in whole or in part, in a base specific manner to a complementary strand. Probes and primers include polypeptide nucleic acids, such as those described in Nielsen et al. (1991) Science 254:1497-1500.

In particular, the term “primers” refers to a single-stranded nucleic acid that can act as a point of initiation of template directed DNA synthesis, such as PCR. In addition to PCR, other suitable isolation, and amplification methods include, for example, the ligase chain reaction (LCR) (see Wu and Wallace, Genomics, 4:560 (1989), Landegren et al., Science, 241:1077 (1988), transcription amplification (Kwoh et al., Proc. Natl. Acad. Sci., USA, 86:1173 (1989)), self-sustained sequence replication (Guatelli et al., Proc. Natl. Acad. Sci. USA, 87:1874 (1990)) and nucleic acid based sequence amplification (NASBA). The latter two amplification methods involve isothermal reactions based on isothermal transcription that produces both single stranded RNA (ssRNA) and double stranded DNA (dsDNA) as the amplified products in a ratio of approximately 30-100 fold more ssRNA than dsDNA.

PCR reactions can be designed based on the human genome sequence and the associated genomic regions or variants identified in Table 1. For example, where a variant is located in an exon, such exon can be isolated and amplified using primers that are complementary to the nucleotide sequences at both ends of the exon. Similarly, where a variant is located in an intron, the entire intron can be isolated and amplified using primers that are complementary to the nucleotide sequences at both ends of the intron.

In preferred embodiments, a probe or primer contains a region of at least about 10 contiguous nucleotides, preferably about 15 contiguous nucleotides, more preferably about 20 contiguous nucleotides, more preferably about 30 contiguous nucleotides, or more preferably about 50 contiguous nucleotides, that can specifically hybridize to a complementary nucleic acid sequence. In addition, a probe or primer is preferably about 100 or fewer nucleotides, more preferably between 6 and 50 nucleotides, and more preferably between 12 and 30 nucleotides in length.

In order to isolate, amplify and detect the presence of a nucleic acid associated with resistance to liver related disease, a probe or primer or set of such probes or primers may include at least 1 variant, preferably at least 2 variants, more preferably at least 3 variants, or more preferably at least 4 variants associated with resistance to liver related disease as shown in Table 1, or variants in common haplotype blocks with the variants in Table 1. To isolate, amplify and detect the present of a nucleic acid associated with susceptibility to liver related disease, a probe or primer or set thereof preferably includes at least 1 variant, preferably at least 2 variants, more preferably at least 3 variants, or more preferably at least 4 variants associated with susceptibility to liver related disease as shown in Table 1 or any other variants in common haplotype blocks as these variants.

In one embodiment, a probe or primer is at least 70% identical to a contiguous nucleotide sequence or complement thereof, preferably at least 80% identical, more preferably at least 90% identical, even more preferably about 95% identical, or even 100% identical to a contiguous nucleotide sequence or complement thereof. In any embodiment, a primer may be labeled (e.g., radioisotope, fluorescent compound, enzyme, or enzyme co-factor.)

The probes and primers herein can be optionally labeled with, for example, a radioactive, fluorescent, biotinylated or chemiluminescent label. Labeled nucleic acids are useful for detection of a hybridization complex and can be used as probes for diagnostic and screening assays.

Labeled probes can be used in cloning of full-length cDNA or genomic DNA by screening cDNA or genomic libraries. Classical methods of constructing cDNA libraries are taught in Sambrook et al., supra. These methods provide for the production of cDNA from mRNA and the insertion of the cDNA into viral or other expression vectors. Typically, libraries of mRNA comprising poly(A) tails can be produced with poly(T) primers. Similarly, cDNA libraries can be produced using the nucleic acid herein as primers. Libraries of cDNA can be made either from selected tissues (e.g., normal or diseased tissue), or from tissues of a mammal treated with, for example, a pharmaceutical agent. Alternatively, many cDNA libraries are available commercially. In a preferred embodiment, the cDNA library is made from diseased or healthy human liver cells. In another preferred embodiment, members of the cDNA library are larger than a nucleic acid hybridization probe, and preferably contain the whole CDNA native sequence.

Genomic DNA can be isolated in a manner similar to the isolation of full-length cDNA. Briefly, the nucleic acids herein, or fragments, derivatives or complement thereof, can be used to probe a library of genomic DNA. Preferably, a genomic DNA library is obtained from liver cells but this is not essential. Such libraries can be in vectors suitable for carrying large segments of a genome, such as P1 or YAC, as described in detail in Sambrook et al., 9.4-9.30. In addition, genomic sequences can be isolated from human BAC libraries, which are commercially available from Research Genetics, Inc., Huntsville, Ala., USA, for example. As an alternative, full-length cDNA, genomic DNA, or any nucleic acid, fragment, derivative or complement thereof, can be obtained by synthesis.

2. Antisense

Antisense nucleic acids, or mimetics thereof that are complementary, in whole or in part, to one or more nucleic acids associated with resistance or susceptibility to liver related disease. Antisense nucleic acids can be used in diagnostics, prognostics and/or treatment of liver related disease. Antisense nucleic acids hybridize under high stringency conditions to target nucleic acids (associated genomic regions). An antisense nucleic acid can bind RNA to form a duplex or a double stranded DNA to form a triplex, which may be assayed.

Preferably, hybridization of an antisense nucleic acid can act directly to block the translation of mRNA associated with susceptibility to liver related disease by hybridizing to targeted mRNA and preventing protein translation. Absolute complementarity, although preferred, is not required. Antisense nucleic acids complementary to non-coding target nucleic acids associated with susceptibility to liver related disease can be used in a similar manner to inhibit translation of endogenous mRNA, which is also associated with susceptibility to liver related disease.

While antisense nucleic acids complementary to a coding region sequence could be used, those complementary to the transcribed, untranslated region are most preferred.

Antisense nucleic acids are preferably at least 10 nucleotides in length, more preferably at least 20 nucleotides, even more preferably at least 40 nucleotides in length, or more preferably at least 80 nucleotides in length.

An antisense nucleic acid can be labeled for convenient detection, such as by using a radioisotope, fluorescent compound, enzyme or an enzyme co-factor.

Regardless of the choice of target sequence, it is preferred that in vitro studies be first performed to quantitate the ability of the antisense nucleic acid to inhibit mRNA expression. It is preferred that these in vitro studies utilize controls that distinguish between antisense inhibition and nonspecific biological effects of nucleic acids in a sample. Additionally, it is envisioned that results obtained using the antisense nucleic acid be compared with those obtained using a control nucleic acid. A control nucleic acid is preferably of approximately the same length as the test antisense nucleic acid and differs from the antisense nucleic acid sequence no more than is necessary to prevent specific hybridization to the target sequence.

The antisense nucleic acids herein can be modified at the base moiety, sugar moiety or phosphate backbone to improve stability of the molecule. Furthermore, the antisense nucleic acids may be hybridized or conjugated to another molecule (e.g., a peptide, hybridization triggered cross-linking agent, cleavage agent or transport agent) for targeting in a host cell or to facilitate the transport across the cell membrane (see, e.g., Letsinger et al. (1989) Proc. Natl. Acad. Sci. USA 86:6553-6556; Lemaitre et al., (1987), Proc. Natl. Acad. Sci. USA 84:648-652); for blood-brain barrier (see, e.g., PCT Publication No. W089/10134); to facilitated the hybridization-triggered cleavage agents (see, e.g., Krol et al. (1988) BioTechniques 6:958-976) or intercalating agents (see, e.g., Zon, (1988), Pharm. Res. 5:539-549).

The antisense nucleic acids may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmenthyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine.

The antisense nucleic acid may also comprise at least one modified sugar moiety selected from the group including but not limited to arabinose, 2-fluoroarabinose, xylulose, and hexose.

In yet another embodiment, the antisense nucleic acid comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense nucleic acid is an alpha.-anomeric oligonucleotide. An .alpha.-anomeric oligonucleotide forms specific double-stranded hybrids with complementary RNA in which, contrary to the usual .beta.-units, the strands run parallel to each other (Gautier, et al., (1987) Nucl. Acids Res. 15:6625-6641). The oligonucleotide is a 2′-0-methylribonucleotide (Inoue, et al., (1987) Nucl. Acids Res. 15:6131-6148), or a chimeric RNA-DNA analogue (Inoue, et al., (1987) FEBS Lett. 215:327-330).

Antisense nucleic acid (as well as other nucleic acids) herein may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein, et al. (1988) Nucl. Acids Res. 16:3209, methylphosphonate oligonucleotides can be prepared by use of controlled pore glass polymer supports Sarin, et al., (1988) Proc. Natl. Acad. Sci. USA 85:7448-7451, etc. Alternately, an antisense nucleic acid can be produced biologically by placing a target nucleic acid in an expression vector in an antisense orientation or by using reverse transcriptase along with other reagents to construct the complementary DNA stand.

Antisense nucleic acids should be delivered to cells that express the sequence in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies which specifically bind receptors or antigens expressed on the target cell surface) can be administered systemically.

A preferred approach to achieve intracellular concentrations of an antisense sufficient to suppress translation of endogenous mRNAs utilizes a recombinant DNA construct in which the antisense oligonucleotide is placed under the control of a strong promoter (e.g., pol III or pol II). The use of such a construct to transfect target cells in a patient will result in the transcription of sufficient amounts of single stranded RNAs which will form complementary base pairs with the endogenous sequence transcripts and thereby prevent translation of the mRNA sequence. For example, a vector can be introduced e.g., such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, (1981) Nature 290:304-310), the promoter contained in the 3′-long terminal repeat of Rous sarcoma virus (Yamamoto, et al., (1980) Cell 22:787-797), the herpes thymidine kinase promoter (Wagner, et al., (1981) Proc. Natl. Acad. Sci. USA. 78:1441-1445), the regulatory sequences of the metallothionein gene (Brinster, et al., (1982) Nature 296:39-42). Any type of plasmid, cosmid, YAC or viral vector can be used to prepare the recombinant DNA construct that can be introduced directly into the tissue site. Alternatively, viral vectors can be used that selectively infect the desired tissue, in which case administration may be accomplished by another route (e.g., systemically).

In any of the embodiments herein, it may be necessary to compare the nucleotide sequence of the nucleic acid obtained, isolated, amplified, or cloned with that of a control. The percent identity of two nucleotide sequences can be determined, for example, by aligning the sequences for optimal comparison purposes. The nucleotides at corresponding positions are compared and the percent identity between the two sequences is a function of the number of identical positions shared by the sequences (e.g., percent identity=the number of identical positions/total number of positions×100). In some embodiments, the length of a sequence aligned for comparison purposes is at least 30%, preferably at least 40%, more preferably at least 60%, and even more preferably at least 70%, 80%, or 90% of the length of the reference sequence or a full sequence gene. An actual comparison of two nucleic acid sequences can be accomplished by well-known methods, for example, using a mathematical algorithm. In one example, such a mathematical algorithm is described in Karlin et al., (1993) Proc. Natl. Acad. Sci. USA, 90:5873-5877. In another example, such mathematical algorithm is the algorithm of Myers and Miller, (1989) CABIOS. Additional algorithms for sequence analysis are known in the art and include ADVANCE and ADAM as described in Torellis and Robotti (1994) Comput. Appl. Biosci., 10:3-5 and FASTA described in Pearson and Lipman (1988) Proc. Natl. Acad. Sci. USA, 85:2444-8.

3. Ribozymes, Knock-Outs and Triple Helix.

Ribozyme molecules designed to catalytically cleave target mRNA transcripts can also be used to prevent translation of such mRNA. See, e.g., PCT Publication No. WO 90/11364; Sarver, et al., (1990) Science 247: 1222-1225.

Ribozymes are enzymatic RNA molecules capable of catalyzing the specific cleavage of RNA. See Rossi, (1994) Current Biology 4:469-471. The mechanism of ribozyme action involves sequence specific hybridization of the ribozyme to complementary target RNA, followed by an endonucleolytic cleavage event. The composition of ribozyme molecules must have one or more sequences complementary to the target mRNA and must include the well known catalytic sequence responsible for MRNA cleavage. See, e.g., U.S. Pat. No. 5,093,246.

While ribozymes that cleave mRNA at site-specific recognition sequences can be used to destroy target mRNAs, the use of hammerhead ribozymes is preferred. Hammerhead ribozymes cleave mRNAs at locations dictated by flanking regions which form complementary base pairs with the target mRNA. The sole requirement is that the target mRNA have the following sequence of two bases: 5′-UG-3′. The construction and production of hammerhead ribozymes are well known in the art and are described in Myers, “Molecular Biology and Biotechnology: A Comprehensive Desk Reference,” (VCH Publishers, New York, 1995) page 833; and in Haseloff and Gerlach, (1988), Nature 334:585-591.

Preferably a ribozyme is engineered so that the cleavage recognition site is located near the 5-end of the target MRNA, i.e., to increase efficiency and minimize the intracellular accumulation of non-functional mRNA transcripts.

The ribozymes herein may further include RNA endoribonucleases, also known as “Cech-type ribozymes,” such as the one which occurs naturally in Tetrahymena thermophila (known as the IVS, or L-19 IVS RNA) and which has been extensively described in Zaug, et al., (1984) Science 224:574-578; Zaug and Cech, (1986) Science 231:470-475; Zaug, et al., (1986) Nature 324:429-433; PCT Publication No. WO 88/04300; Been and Cech, (1986) Cell 47:207-216.

As in the antisense approach, ribozymes can be composed of modified nucleic acids (e.g., for improved stability, targeting, etc.) and are preferably delivered to cells that express the target gene in vivo. A preferred method of delivery involves using a DNA construct encoding the ribozyme under the control of a strong constitutive promoters (e.g., pol III or pol II), so that transfected cells will produce sufficient quantities of the ribozyme to destroy endogenous target mRNA and inhibit translation. Because ribozymes, unlike antisense molecules, are catalytic, a lower intracellular concentration is required for efficiency.

Endogenous target gene expression can also be reduced by inactivating or “knocking out” the target nucleic acid (e.g., coding regions or regulatory regions of the target gene) using targeted homologous recombination. See Smithies, et al., (1985) Nature 317:230-234; Thomas and Capecchi, (1987) Cell 51:503-512; Thompson, et al., (1989) Cell 5:313-321. For example, a non-functional nucleic acid (or a completely unrelated DNA sequence) flanked by DNA homologous to the endogenous target nucleic acid can be used, with or without a selectable marker and/or a negative selectable marker, to transfect cells which express the target gene in vivo. Insertion of the DNA construct, via targeted homologous recombination, results in inactivation of the target gene. Such approaches can be used in humans provided the recombinant DNA constructs are directly administered or targeted to the required site in vivo using appropriate viral vectors.

Alternatively, endogenous expression of a target gene can be reduced by targeting deoxyribonucleotide sequences complementary to the regulatory region of the target gene (i.e., the target gene promoter and/or enhancers) to form triple helical structures which prevent transcription of the target gene in target cells in the body. See generally, Helene, (1991), Anticancer Drug Des., 6(6):569-584; Helene, et al., (1992), Ann. N.Y. Acad. Sci., 60:27-36; and Maher, (1992), Bioassays 14(12):807-815.

Nucleic acids to be used in triple helix formation for the inhibition of transcription should be single stranded and composed of deoxynucleotides. The base composition of these oligonucleotides must be designed to promote triple helix formation via Hoogsteen base pairing rules, which generally require sizable stretches of either purines or pyrimidines to be present on one strand of a duplex. Nucleic acids may be pyrimidine-based, which will result in TAT and CGC.sup.+ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen which are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so-called “switchback” nucleic acid. Switchback nucleic acids are synthesized in an alternating 5′-3′, 3′-5′ manner, such that they base pair with first one strand of a duplex and then the other, eliminating the necessity for a sizable stretch of either purines or pyrimidines to be present on one strand of a duplex.

In instances wherein the antisense, ribozyme, “knock-out,” and/or triple helix molecules described herein are utilized to inhibit a variant gene expression (e.g., expression of nucleic acids associated with susceptibility to liver related disease), it is possible that the technique may so efficiently reduce or inhibit the transcription (triple helix; knock-out) and/or translation (antisense, ribozyme) of mRNA that it may cause severe negative side effects. (For example, knocking out all Bcl-2 functions results in apoptosis). In such cases, to ensure that substantially normal levels of target gene products or desired gene products are maintained, nucleic acids which encode and polypeptides exhibiting a desired target gene activity (e.g., polypeptides associated with resistance to liver related disease) may, be introduced into cells via gene therapy methods. The desired gene product should not contain sequences susceptible to antisense, ribozyme or triple helix treatments that are being utilized.

The antisense, ribozyme, and triple helix molecules herein may be prepared by any method known in the art for the synthesis of DNA and RNA molecules.

4. Expression Vectors and Vectors

Any one or more of the nucleic acids herein can be inserted into a vector. A vector can be used, for example, to transfer nucleic acids or to express the inserted nucleic acids. In one embodiment, nucleic acids comprising an exon associated genomic region of Bcl-2 can be inserted into an expression vector to express a partial or complete Bcl-2 gene product. An exon associated genomic region can be in the coding region or outside the coding region. Expression vectors may be constructed using methods known in the art. Such methods include in vitro recombinant DNA techniques, synthetic techniques, in vivo genetic recombination, and other techniques described in Sambrook, J. et al. “Molecular Cloning, A Laboratory Manual,” (Cold Spring Harbor Press, Plainview, N.Y. 1989), and Ausubel, F. M. et al. “Current Protocols in Molecular Biology”, (John Wiley & Sons, New York, N.Y., 1989)

There are multiple types of expression vectors. One type of expression vector is a plasmid, which refers to a circular double stranded DNA molecule into which additional DNA segments can be ligated. Another type of vector is a viral vector, wherein additional DNA segments can be ligated into a viral genome. Viral vectors include replication defective retroviruses, adenoviruses and adeno-associated viruses. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. A preferable expression vector is either a plasmid or a viral vector.

The expression vectors herein can include one or more regulatory sequences, selected on the basis of the host cells to be used and the level of expression desired. The regulatory sequences can be operably linked to the nucleic acid sequence to be expressed. The term operably linked refers to a nucleic acid of interest that is linked to one or more regulatory sequences in a manner that allows for the expression of the nucleic acid of interest. The term regulatory sequence is intended to include promoters, enhancers and other expression control elements (e.g., polyadenylation signals). Such regulatory sequences are described, for example, in Goeddel, “Gene Expression Technology: Methods in Enzymology” (1990) 185, Academic Press, San Diego, Calif. Regulatory sequences include those which direct constitutive expression of a nucleotide sequence in many types of host cell and those which direct expression of the nucleotide sequence only in certain host cells (e.g., tissue-specific regulatory sequences).

In another embodiment, a coding region of an associated genomic region can be inserted into an expression vector with or without a non-coding nucleic region of interest. The difference in expression or activity between a vector comprising both the non-coding and coding sequence can be detected using methods known in the art.

The vectors herein can be inserted into a host cell. The term “host cell” refers not only to a particular subject cell but also to the progeny or potential progeny of such a cell. Because certain modifications may occur in succeeding generations due to either mutations or environmental influences, such progeny may not, in fact, be identical to cells, but are still included within the scope of the term as used herein.

Vectors can be introduced into prokaryotic or eukaryotic cells via conventional transformation or transfection techniques. For example, expression systems in bacteria include those descried in Chang et al., (1978) Nature 275:615, and Siebenlist et al., (1980) Cell 20:269; expression systems in yeast include those described in Kelly and Hynes, EMBO J. (1985) 4:475-479; expression systems in insect cells include those described in Maeda et al., (1985) Nature 315:592-594 and expression in mammalian cells inflammatory disease described, for example, in Dijkema et al., (1985) EMBO J. 4:761. Vector constructs can comprise either sense or antisense sequences, or both.

As used herein, the terms transformation and transfection are intended to refer to a variety of art-recognized techniques for introducing a foreign nucleic acid molecule (e.g., DNA) into a host cell, including calcium phosphate or calcium chloride co-precipitation, DEAF-dextran-mediated transfection, lipofection, or electroporation. Suitable methods for transforming or transfecting host cells can be found in Sambrook, et al. and other laboratory manuals. For stable transfection of mammalian cells, it is known that, depending upon the expression vector and transfection technique used, only a small fraction of cells may integrate the foreign DNA into their genome. In order to identify and select these integrants, a gene that encodes a selectable marker is generally introduced into the host cells along with the gene of interest. Preferred selectable markers include those that confer resistance to drugs. Nucleic acid molecules encoding a selectable marker can be introduced into a host cell on the same vector as the nucleic acids or can be introduced on a separate vector. Cells stably transfected with the introduced nucleic acid molecule can be identified by drug selection (e.g., cells that have incorporated the selectable marker gene will survive, while the other cells die).

Host cells can be used to produce polypeptides encoded by any of the nucleic acids herein. Suitable host cells and methods for producing polypeptides using such host cells are discussed in Goeddel, supra. For large scale protein production, a unicellular organism such as E. coli, baculovirus vectors, or cells of higher organisms such as vertebrates, particularly mammals, e.g. COS7 cells, may be useful. In some situations, it may be desirable to express a gene in a eukaryotic cell where the gene will benefit from native folding and posttranslational modifications. Host cells into which an expression vector has been introduced may be cultured in suitable medium such that the polypeptide is produced. The polypeptide herein may be isolated from the medium or from the host cell.

Host cells can also be used to produce nonhuman transgenic animals. For example, in one embodiment, a host cell is a fertilized oocyte or an embryonic stem cell into which a nucleic acid (e.g., an exogenous liver related disease gene or a nucleic acid encoding a polypeptide herein) has been introduced. Such host cells can then be used to create non-human transgenic animals in which exogenous nucleotide sequences have been introduced into the genome or homologous recombinant animals in which endogenous nucleotide sequences have been altered. Such animals are useful for studying the function and/or activity of the nucleotide sequence and polypeptide encoded by the sequence and for identifying and/or evaluating modulators of their activity. As used herein, a “transgenic animal” is a non-human animal, preferably a mammal, more preferably a rodent such as a rat or mouse, in which one or more of the cells of the animal include a transgene. Other examples of transgenic animals include, for example, non-human primates, sheep, dogs, cows, goats, chickens and amphibians. A transgene is an exogenous DNA which is integrated into the genome of a cell from which a transgenic animal develops and which remains in the genome of the mature animal, thereby directing the expression of an encoded gene product in one or more cell types or tissues of the transgenic animal. As used herein, an homologous recombinant animal is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

Methods for generating transgenic animals via embryo manipulation and microinjection, particularly animals such as mice, are conventional in the art and are described, for example, in U.S. Pat. Nos. 4,736,866, 4,870,009, 4,873,191 and in Hogan, “Manipulating the Mouse Embryo,” (Cold Spring Harbor Laboratory Press, Cold Spring Harbor, N.Y., 1986). Methods for constructing homologous recombination vectors and homologous recombinant animals are described further in Bradley (1991) Current Opinion in BioTechnology, 2:823-829. Clones of the non-human transgenic animals described herein can also be produced according to the methods described in Wilmut et al. (1997) Nature 385:810-813; and PCT Publication Nos. WO 97/07668 and WO 97/07669.

III. Polypeptides

Liver related disease polypeptides such as those enoded by the associated genomic regions herein are useful in the prognostics, diagnostics, prevention, treatment and study of liver related disease. Such polypeptides may be naturally occurring or recombinantly produced using methods known in the art.

A liver related disease polypeptide can be associated with resistance or susceptibility to liver related disease. A polypeptide associated with resistance to liver related disease may be one that is differentially expressed in individuals having a phenotype of resistance to liver related disease or one that is regulated or encoded in whole or in part by a nucleic acid associated with resistance to liver related disease. In one example, a polypeptide associated with liver related disease can be recombinantly produced using an expression vector having a non-coding regulatory region associated with resistance to liver related disease, operably linked to a liver related disease polypeptide. The expression vector is introduced into a host cell under conditions appropriate for expression. The polypeptide can then be isolated from the host cell using standard protein purification techniques.

Similarly, a polypeptide associated with susceptibility to liver related disease may be one that is differentially expressed in individuals having a phenotype of susceptibility to liver related disease (e.g., cirrhosis of the liver) or one that is regulated or encoded, in whole or in part, by nucleic acids associated with susceptibility to liver related disease. For example, a polypeptide associated with liver related disease can be recombinantly produced by introducing an expression vector with a coding nucleic acid associated with susceptibility to liver related disease into a host cell. The host cell is maintained under conditions suitable for expression. The polypeptide is then isolated from the host cell.

In one embodiment, a polypeptide associated with resistance to liver related disease can be produced by inserting a non-coding nucleic acid or nucleic acid outside coding region which is associated with resistance to liver related disease, operably linked to an associated genomic region coding sequence, into a host cell under conditions appropriate for protein synthesis, and then purifying the polypeptide expressed by the host cell

A similar method can be used to produce a polypeptide associated with susceptibility to liver related disease. For example, a non-coding nucleic acid or nucleic acid outside coding region which is associated with susceptibility to liver related disease, operably linked to an associated genomic region coding sequence, can be inserted into a host cell under conditions appropriate for protein synthesis. The resulting polypeptide associated with susceptibility to liver related disease is then collected and purified.

In a preferred embodiment, a polypeptide associated with susceptibility to liver related disease can be produced by inserting a vector comprising a coding nucleic acid associated with susceptibility or resistance to liver related disease and then purifying the polypeptide expressed by the host cell.

In preferred embodiments, the polypeptides are purified. There are various degrees of purity. While a polypeptide can be purified to homogeneity, preparations in which a polypeptide is not purified to homogeneity are also useful where the polypeptide retains a desired function even in the presence of considerable amount of other components. In some embodiments, polypeptides are substantially free of cellular material which includes preparations of a polypeptide having less than about 30% (dry weight) other polypeptides (e.g., contaminating polypeptides), less than about 20% other polypeptides, less than about 10% other polypeptides, or less than about 5% other polypeptides.

When a polypeptide is recombinantly produced, it can also be substantially free of culture medium. In preferred embodiments, culture medium represents less than about 20% of the volume of the polypeptide preparation, preferably less than about 10% of the volume of the polypeptide preparation or more preferably less than about 5% of the volume of the polypeptide preparation. Polypeptides that are substantially free of chemical precursors or other chemicals generally include those that are separated from chemicals that are involved in its synthesis. In one embodiment, the polypeptides are substantially free of chemical precursors or other chemicals such that a preparation of the polypeptides has less than about 30% (dry weight) chemical precursors or other chemicals, preferably less than about 20% chemical precursors or other chemicals, more preferably less than about 10% chemical precursors or other chemicals or more preferably less than about 5% chemical precursors or other chemicals.

As used herein, two polypeptides are substantially homologous when their amino acid sequences are at least about 45% homologous, or preferably at least about 75% homologous, or more preferably at least about 85% homologous, or even more preferably greater than about 95% homologous. To determine the percent homology of two polypeptides, the amino acid sequences are aligned for optimal comparison purposes. The amino acid residues at corresponding positions are compared. The percent homology between two amino acid sequences is a function of the number of identical positions shared by the sequences (e.g., percent homology equals the number of identical positions/total number of positions times 100).

Some polypeptides (e.g., synonymous or conservative variants) may have a lower degree of sequence homology but are still able to perform one or more of the same functions. Conservative substitutions that can maintain the same function include replacements among aliphatic amino acids methionine, valinel, leucine and isoleucine; interchange of the hydroxyl residues serine and threonine; exchange of acidic residues aspartic and glutamic acids; substitution between amide residues asparagine and glutamine, exchange between basic residues lysine and arginine, and replacements among aromatic residues phenylalanin, tyrosine and tryptophan. Alanine and glycine may also result in conservative substitutions.

Other polypeptides that may not be able to perform one or more of the same functions may be variants containing one or more non-conservative amino acid substitutions or deletions, insertions, inversions or substitution of one or more amino acid residues. Amino acids that are essential for function of a polypeptide can be identified by various methods known in the art, such as site-directed mutagenesis or alanine-scanning mutagenesis. See Cunningham et al., (1989) Science, 244:1081-1085. The latter procedure can introduce a single alanine mutation at every residue in the molecule. The resulting variants are then tested for biological activity in vitro or in vivo. Residues that are critical for polypeptide activity or inactivity are identified by comparing the two variants (with and without the alanine mutation). Polypeptide activity can also be determined by structural analysis such as crystallization, nuclear magnetic resonance or photoaffinity labeling. See Smith et al, (1992) J. Mol. Biol., 224:899-904; and de Vos et al. (1992) Science, 255:306-312.

1. Fusion Proteins

Any polypeptides herein can be made part of a fusion protein. The term “fusion protein” or “fusion polypeptide” refers to a liver related disease polypeptide (a polypeptide associated with resistance or susceptibility to liver related disease) operatively linked to a non-liver related disease polypeptide or a heterologous polypeptide having an amino acid sequence not substantially homologous to a liver related disease amino acid sequence. “Operatively linked” indicates that the polypeptide and the heterologous protein are fused, for example, the non-liver related disease polypeptide can be fused to the N-terminus or C-terminus of the liver related disease polypeptide. In a preferred embodiment, the fusion polypeptide does not affect the function of the liver related disease polypeptide. Examples of fusion polypeptide that do not affect the function of a polypeptide include a GST-fusion polypeptides in which the liver related disease polypeptide sequences are fused to the C-terminus of the GST sequences. Other types of fusion polypeptides, include enzymatic fusion polypeptides, for example β-galactosidase fusions, yeast two-hybrid GAL fusions, poly-His fusions and Ig fusions. Fusion polypeptides, especially poly-His fusions, can facilitate the purification of recombinant polypeptide. In some host cells, such as mammalian cells, expression and secretion of a liver related disease polypeptide can be increased using a heterologous signal sequence. Therefore, in a preferred embodiment, a liver related disease polypeptide may be fused to a heterologous signal sequence at its N-terminus. In another embodiment, a fusion protein may comprise of a liver related disease polypeptide and various portions of immunoglobulin constant regions such as the Fc portion. Fe portions are useful in therapy and diagnosis and may result in improved pharmacokinetic properties. Fc portions can also be used in high-throughput screening assays to identify binding molecules, agonists and antagonists. See, e.g., Bennett et al.; J. of Molec. Recog., (1995) 8:52-58 and Johanson et al., (1995) J. of Biol. Chem., 270,16:9459-9471. In a preferred embodiment, soluble fusion proteins comprise of a liver related disease polypeptide and one or more of the constant regions of heavy or light chains of immunoglobulins (e.g. IgG, IgM, IgA, IgD, IgE).

A fusion protein can be produced by standard recombinant DNA techniques as described herein. For example, DNA fragments coding for the different polypeptide sequences are ligated together in accordance with conventional techniques. The fusion gene can be synthesized by conventional techniques such as automated DNA synthesizers. Alternatively, PCR amplification of nucleic acid fragments can be carried out using anchor primers which give rise to complementary overhangs between two consecutive nucleic acid fragments that can subsequently be annealed and reamplified to generate a chimeric nucleic acid sequence. Moreover, many expression vectors are commercially available that already encode a fusion moiety (e.g., a GST protein). A nucleic acid encoding a polypeptide herein can be cloned into such an expression vector such that the fusion moiety is linked in-frame to the polypeptide.

2. Antibodies

Any of the polypeptides herein, or fragments, derivatives, or complements thereof, can be used as an immunogen (e.g. epitope) to generate polypeptide-specific antibodies. Antibodies can be used to detect, isolate and inhibit the activity of one or more liver related disease polypeptides.

To generate liver related disease antibodies, a liver related disease polypeptide or a fragment thereof is used as an epitope. In preferred embodiments, an epitope is at least 6 amino acids, at least 9 amino acids, at least 20 amino acids, at least 40 amino acids, or at least 80 amino acids in length. The epitope or polypeptide fragment preferably comprises a domain, segment or motif that can be identified by analysis using well-known methods, for example, signal polypeptides, extracellular domains, transmembrane segments or loops, ligand binding regions, zinc finger domains, DNA binding domains, acylation sites, glycosylation sites or phosphorylation sites.

Examples of antibodies include polyclonal, monoclonal, humanized, chimeric, single chain antibodies, Fab fragments, F(ab′)₂ fragments, fragments produced by FAb expression library, anti-idiotypic (anti-Id) antibodies or epitope-binding fragments of any of the above.

Polyclonal antibodies are prepared by immunizing a suitable subject (e.g., goats, rabbits, rats, mice or humans) with a desired antigen. The antibody titer in the immunized subject can be monitored over time using methods known in the art, such as by using an enzyme linked immunosorbent assay (ELISA). The antibodies can then be isolated from the subject (e.g., from blood) and further purified using techniques, such as protein A chromatography, to obtain the IgG fraction.

At an appropriate time after immunization, such as when the antibody titers are highest, antibody-producing cells can be obtained from the subject and used for the preparation of monoclonal antibodies. Monoclonal antibodies are populations of antibodies that contain only one species of an antigen-binding site and are capable of immunoreacting with only one particular epitope of liver related disease polypeptides. A monoclonal antibody composition, therefore, typically displays a single binding affinity for a particular polypeptide with which it immunoreacts.

There are numerous methods known in the art for producing monoclonal antibodies. In one example, monoclonal antibodies can be obtained by fusing individual lymphocytes (typically splenocutes) from an immunized animal (typically a mouse or a rat) with cells derived from an immortal B lymphocyte tumor (typically a myeloma) to produce a hybridoma. The culture supernatants of the resulting hybridoma cells are screened to identify a hybridoma producing a monoclonal antibody that specifically binds to a polypeptide of interest. Other techniques for producing hybridoma include the human B cell hybridoma technique described in Kozbor et al. (1983) Immunol. Today, 4:72; the EBV-hybridoma technique and the trioma techniques.

Alternatively, monoclonal antibodies can be identified and isolated by screening a combinatorial immunoglobulin library, such as an antibody phage display library. The library can be screened with one or more of the polypeptides herein. Identified members are then isolated using techniques known in the art. Kits for generating and screening phage display libraries are commercially available. See for example, the Pharmacia Recombinant Phage Antibody System, Catalog No. 27-9400-01, and the Stratagene SurjZAPTM Phag & Display Kit, Catalog No. 240612. Other methods and reagents for generating and screening antibody display libraries are disclosed in PCT Publication No. WO 92/01047; PCT Publication No. WO 90/02809; Fuchs et al. (1991) Bio/Technology, 9:1370-1372; Hay et al. (1992) Hum. Antibod. Hybridomas, 3:81-85; Huse et al. (1989) Science 246:1275-1281; Griffith et al. (1993) EMBO J. 12:725-734.

The monoclonal antibodies are chimeric and humanized. Humanized monoclonal antibodies can be obtained using standard recombinant DNA techniques in which the variable region genes (e.g., of a rodent antibody), are cloned into a mammalian expression vector containing the appropriate human light change and heavy chain region genes. In this example, the resulting chimeric monoclonal antibodies has the antigen-binding capacity from the variable region of the rodent but is significantly less immunogenic because of the humanized light and heavy chain regions. See, e.g., Surender K. Vaswani, Ann. (1998) Allergy Asthma. Immunol. 81:105-119.

Any of the antibodies can further be coupled to a substance (label) for detection of a polypeptide-antibody binding complex. Examples of labels include, enzymes, prosthetic groups, fluorescent materials, luminescent materials, bioluminescent materials, or radioactive materials. Examples of suitable enzymes include, for example, horseradish peroxidase, alkaline phosphatase, β-galactosidase, or acetylcholinesterase. Examples of suitable prosthetic group complexes include, for example, streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin. An example of a luminescent material is luminol. Examples of bioluminescent materials include luciferase, luciferin and aequorin. Examples of suitable radioactive material include ₁₂₅I, ₁₃₁I, ₃₅S or ₃H.

The antibodies can be used to isolate one or more liver related disease polypeptides using standard techniques such as affinity chromatography or immunoprecipitation. The antibodies can also be used to detect the presence or absence of a particular polypeptide (e.g., a polypeptide associated with resistance or susceptibility to liver related disease) in a cell, cell lysate, cell supernatant, tissue sample or elsewhere. Preferably, the antibodies can further be used to inhibit or suppress the activity of such polypeptides by specifically binding to the polypeptides.

IV. Diagnostic And Prognostic Assays

The nucleic acids, polypeptides, antibodies and other compositions herein may be utilized as reagents (e.g., in pre-packaged kits) for prognosis and diagnosis of susceptibility or resistance to liver related disease, in particular cirrhosis. A variety of methods may be used to prognosticate and diagnose susceptibility or resistance to liver related disease. The following methods are provided as examples and not as limitations of means to diagnose liver related disease.

1. Detection of Liver Related Disease Nucleic Acids

Detection of presence or increased level of one or more nucleic acids, or fragments, derivatives, variants or complements thereof, associated with resistance to liver related disease is a prognostic and diagnostic for resistance to liver related disease. On the other hand, detection of presence or increased level of one or more nucleic acids, or fragments, derivatives, variants or complements thereof, associated with susceptibility to liver related disease is a prognostic and diagnostic for susceptibility to liver related disease.

Detection of nucleic acids may be made using any method known in the art, for example, Southern or Northern analyses, in situ hybridizations analyses, single stranded conformational polymorphism analyses, polymerase chain reaction analyses and nucleic acid microarray analyses. Such analyses may reveal both quantitative and qualitative aspects of the expression pattern of liver related disease polypeptides. In particular, such analyses may reveal expression patterns or polypeptides associated with resistance or susceptibility to liver related disease.

In one example, a diagnosis or prognosis is made using a test sample containing genomic DNA or RNA obtained from the individual to be tested. The individual can be an adult, child or fetus. In a preferred embodiment, the individual is a human. The test sample can be from any source which contains genomic DNA or RNA, including for example, blood, amniotic fluid, cerebrospinal fluid, skin, muscle, buccal or conjunctival mucosa, placenta, gastrointestinal tract or other organs. In a preferred embodiment a DNA or RNA sample is obtained from liver cells or liver tissue. A test sample of DNA from fetal cells or tissue can be obtained by appropriate methods such as by amniocentesis or chorionic villus sampling. The test sample is subjected to one or more tests to identify the presence or absence of a nucleic acid of interest.

In one embodiment, Southern blot, northern blot or similar analyses methods are used to identify the presence or absence of genomic DNA sequence using complementary nucleic acid probes associated with resistance to liver related disease. The nucleic acid probes are preferably labeled before contacted with a hybridization sample.

In hybridization analysis, the hybridization sample is maintained under conditions sufficient to allow for specific hybridization of the nucleic acid probe to the target nucleic acid. In a preferred embodiment, the labeled nucleic acid probe and target nucleic acid specifically hybridize with no mismatches. Specific hybridization can be performed under stringent conditions disclosed herein and can be detected using standard methods. Hybridization is indicative of the presence or absence of a target nucleic acid. Specific hybridization to a nucleic acid or variant associated with resistance to liver related disease is a diagnostic for resistance to liver related disease. Specific hybridization to a nucleic acid or variant associated with susceptibility to liver related disease is a diagnostic for susceptibility to liver related disease. More than one probe can be used concurrently.

In a preferred embodiment, a nucleic acid probe is an allele-specific probe. See Saild, R. et al., (1986) Nature 324:163-166. Allele-specific probes can used to identify the presence or absence of one or more variants in a test sample of DNA obtained from an individual. A target nucleic acid is amplified using any method herein. Flanking sequences may also be amplified. In the case of Southern analysis, the amplified target nucleic acid is dot-blotted, using standard methods and the blot is then contacted with an allele specific nucleic acid probe. See Ausubel, F. el al., “Current Protocols in Molecular Biology” (eds. John Wiley & Sons). Detection of specific hybridization of an allele-specific probe to a target nucleic acid associated with resistance to liver related disease is a diagnostic for resistance to liver related disease. Detection of specific hybridization of an allele-specific probe to a target nucleic acid associated with susceptibility to liver related disease is a diagnostic for susceptibility to liver related disease.

Allele-specific probes are nucleic acids, mimetics, or a combination thereof, of approximately 10-50 base pairs or more preferably approximately 15-30 base pairs that specifically hybridize to one or more target nucleic acids. Target nucleic acids are any of the nucleic acids herein.

In one example, a target nucleic acid is a nucleic acid associated with resistance to liver related disease. Nucleic acid probes or sets or kits thereof (whether for Southern analysis, or other nucleic acid analysis techniques herein) may include one or more variants associated with resistance to liver related disease, more preferably two or more variants associated with resistance to liver related disease, more preferably three or more variants associated with resistance to liver related disease or more preferably four or more variants associated with resistance to liver related disease.

In another example, a target nucleic acid is a nucleic acid associated with susceptibility to liver related disease. Nucleic acid probes or sets or kits thereof (whether for Southern analysis, or other nucleic acid analysis techniques herein) may include on or more variants associated with susceptibility to liver related disease, more preferably two or more variants associated with susceptibility to liver related disease, more preferably three or more variants associated with susceptibility to liver related disease, or more preferably four or more variants associated with susceptibility to liver related disease. An allele-specific nucleic acid can be prepared using standard methods.

Another method for detecting nucleic acids associated with resistance or susceptibility to liver related disease is Northern analysis. Northern analysis can be used to identify gene expression patterns (e.g., mRNA) of liver related disease polypeptides. See Ausubel, F. el al., “Current Protocols in Molecular Biology” (eds. John Wiley & Sons 1999). For Northern analysis, a test sample of RNA is obtained from an individual by appropriate means. Specific hybridization of a nucleic acid probe that is complementary to the RNA sequence encoding a polypeptide associated with resistance to liver related disease is a diagnostic for resistance to liver related disease. Specific hybridization of a nucleic acid probe to the RNA sequence encoding a polypeptide associated with susceptibility to liver related disease is a diagnostic for susceptibility to liver related disease. A nucleic acid probe is preferably labeled. A nucleic acid probe is preferably an allele-specific probe to one or more of the variants described in Table 1, or may include kits or collections of probes with more than one of such probes.

Alternative diagnostic and prognostic methods employ amplification of target nucleic acids associated with resistance or susceptibility to liver related disease, e.g., by PCR. This is especially useful for the target nucleic acids present in very low quantities. In one embodiment, amplification of target nucleic acid probes associated with resistance to liver related disease indicates their presence and is a prognostic and diagnostic of resistance to liver related disease. Amplification of target nucleic acids associated with susceptibility to liver related disease indicates their presence and is a prognostic and diagnostic of susceptibility to liver related disease.

In another embodiment, CDNA is obtained from target RNA nucleic acids by reverse transcription. Nucleic acid sequences within the CDNA are then used as templates for amplification reactions. Nucleic acids used as primers in the reverse transcription and amplification reaction steps can be chosen from any of the nucleic acids herein. For detection of amplified products, the nucleic acid amplification may be performed using labeled nucleic acids. Alternatively, enough amplified product may be made such that the product may be visualized by standard ethidium bromide staining or by utilizing other suitable nucleic acid staining method.

Microarrays can also be utilized for diagnosis and prognosis of resistance or susceptibility to liver related disease. Microarrays comprise of probes that are complementary to target nucleic acid sequences from an individual. A microarray probe is preferably allele specific. In one embodiment, the microarray comprises a plurality of different probes, each coupled to a surface of a substrate in different known locations and each, capable of binding complementary strands. See, e.g., U.S. Pat. No. 5,143,854 and PCT Publication Nos. WO 90/15070 and WO 92/10092. These microarrays can generally be produced using mechanical synthesis methods or light directed synthesis methods that incorporate a combination of photolithographic methods and solid phase oligonucleotide synthesis methods. See Fodor et al., (1991) Science 251:767-777; and U.S. Pat. No. 5,424,186. Techniques for the mechanical synthesis of microarrays are described in, for example, U.S. Pat. No. 5,384,261.

Once a microarray is prepared, a target nucleic acid is hybridized to the microarray before the microarray is scanned. Typical hybridization and scanning procedures are described in PCT Publication Nos. WO 92/10092 and WO 95/11995, and U.S. Pat. No. 5,424,186. Briefly, target nucleic acid sequences that include one or more previously identified variants or polymorphisms are amplified and labeled by well-known amplification techniques, such as PCR. Primers that are complementary to both strands of the target sequence (upstream and downstream from a variant or polymorphism) may be used to amplify the target region. Asymmetric PCR techniques may be used. An amplified target, preferably incorporating a label, is then hybridized with the microarray under appropriate conditions. Upon completion of hybridization and washing of the microarray, the microarray is scanned to determine the position on the microarray to which the target sequence hybridizes. The hybridization data obtained from the scan is typically in the form of fluorescence intensities as a function of location on the microarray.

Although primarily described in terms of a single detection block, such as for the detection of a single polymorphism, microarrays can include multiple detection blocks, and thus be capable of analyzing multiple specific polymorphisms. In an alternative arrangement, detection blocks may be grouped within a single microarray or in multiple separate microarrays so that varying optimal conditions may be used during the hybridization of the target to the microarray. For example, it may be desirable to provide for the detection of polymorphisms that fall within G-C rich stretches of a genomic sequence separately from those that fall in A-T rich segments for optimization of hybridization conditions. Additional description of use of nucleic acid microarrays for detection of polymorphisms can be found, for example, in U.S. Pat. Nos. 5,858,659 and 5,837,832, the entire teachings of which are incorporated by reference herein.

Other methods to detect variant nucleic acids include, for example, direct manual sequencing (Church and Gilbert, (1988) Proc. Natl. Acad. Sci. USA 81:1991-1995; Sanger, F. et al. (1977) Proc. Natl. Acad. Sci. USA 74:5463-5467; and U.S. Pat. No. 5,288,644); automated fluorescent sequencing; single-stranded conformation polymorphism assays; clamped denaturing gel electrophoresis; denaturing gradient gel electrophoresis (Sheffield, V. C. et al. (1981) Proc. Natl. Acad. Sci. USA 86:232-236), mobility shift analysis (Orita, M. et al. (1989) Proc. Natl. Acad. Sci. USA 86:2766-2770), restriction enzyme analysis (Flavell et al. (1978) Cell 15:25; Geever, et al. (1981) Proc. Natl. Acad. Sci. USA 78:5081); heteroduplex analysis; chemical mismatch cleavage (Cotton et al. (1985) Proc. Natl. Acad. Sci. USA 85:4397-4401); RNase protection assays (Myers, R. M. et al. (1985) Science 230:1242); and use of polypeptides which recognize nucleotide mismatches, such as E. coli mutS protein.

2. Detection of Liver Related Disease Polypeptides

Detecting the presence, level of expression, activity and location of liver related disease polypeptides may be used as a diagnostic or prognostic for resistance or susceptibility to liver related disease. Briefly, detection of the presence, level of expression or enhanced activity of polypeptides associated with resistance to liver related disease is a diagnostic and prognostic for resistance to liver related disease. Detection of the presence, level of expression or enhanced activity of polypeptides associated with susceptibility to liver related disease is a diagnostic and prognostic for susceptibility to liver related disease.

Proteins may be analyzed from any tissue or cell type but preferably liver tissue or hepatic cells. Analyses can be made in vivo or in vitro. In a preferred embodiment a biopsy (or tissue sample) is obtained from the liver of an individual to be tested, blood, or the like.

Methods to detect and isolate polypeptides are known to those of skill in the art and include, for example, enzymes linked immunosorbent assays (ELISAs), immunoprecipitations, immunofluorescence, immunoblotting, Western blotting, spectroscopy, colorimetry, electrophoresis and isoelectric focusing. See U.S. Pat. No. 4,376,110; see also Ausubel, F. et al., “Current Protocols in Molecular Biology” (Eds. John Wiley & Sons, chapter 10). Protein detection and isolation methods employed may also be those described in Harlow and Lane (Harlow, E. and Lane, D., “Antibodies: A Laboratory Manual,” Cold Spring Harbor Laboratory Press, Cold Spring Harbor, N.Y., 1998).

In one embodiment, the presence, amount and location of polypeptides associated with resistance to liver related disease can be determined using a probe or an antibody that specifically binds one or more polypeptides associated with resistance to liver related disease. In another embodiment, the presence, absence, amount or location of a polypeptide associated with susceptibility to liver related disease can be determined using a probe or antibody that specifically bind one or more polypeptides associated with susceptibility to liver related disease.

Antibodies, such as those described herein may be used to determine the presence of a polypeptide associated with resistance or susceptibility to liver related disease.

In a preferred embodiment, a probe or antibody is labeled directly or indirectly. Direct labeling involves coupling (physically linking) a detectable substance to an antibody or a probe. Indirect labeling involves the reactivity of the probe with another reagent that is directly labeled. An example of indirect labeling includes, for example, detection of a primary antibody using a fluorescently labeled secondary antibody and end labeling of a DNA probe with biotin such that it can be detected with fluorescently labeled streptavidin.

A solid support may be utilized to immobilize either the antibody or probe or the sample. In one example, a sample may be immobilized onto a solid support such as nitrocellulose, which is capable of immobilizing cells, cell particles, or soluble proteins. The support may then be washed with suitable buffers followed by treatment with a detectably labeled antibody. The amount of bound labeled antibody on the solid support may then be detected by conventional means. Well known supports include glass, polystyrene, polypropylene, polyethylene, dextran, nylon, amylases, natural and modified celluloses, polyacrylamides, gabbros, and magnetite.

The antibodies herein can be linked to an enzyme and used in enzyme immunoassay. See Voller, “The Enzyme Linked Immunosorbent Assay (ELISA)”, Diagnostic Horizons 2:1-7 (Microbiological Associates Quarterly Publication, Walkersville, Md. 1978); Maggio, “Enzyme Immunoassay” (CRC Press, Boca Raton, Fla. 1980); Ishikawa, et al., “Enzyme Immunoassay” (Kgaku Shoin, Tokyo, 1981). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes that can be used to label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Detection can be accomplished by calorimetric methods which employ a chromogenic substrate for the enzyme. Detection can also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect fingerprint gene wild type or mutant peptides through the use of a radioimmunoassay. See Weintraub, B., “Principles of Radioimmunoassays, Seventh Training Course on Radioligand Assay Techniques” (The Endocrine Society, March, 1986). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can be detected due to fluorescence. Among the most commonly used fluorescent labeling compounds are fluorescein isothiocyanate, rhodamine, phycoerythrin, phycocyanin, allophycocyanin, o-phthaldehyde and fluorescamine. The fluorescently labeled antibody can be coupled with light microscopic, flow cytometric or fluorietric detection. In one example, antibodies, or fragments thereof, may be employed histologically, as in immunofluorescence or immunoelectron microscopy, for in situ detection of a polypeptide associated with resistance or susceptibility to liver related disease. In situ detection may be accomplished by removing a histological specimen from a patient, such as by liver biopsy. The specimen is then applied with a labeled antibody described herein. The antibody or fragment is preferably applied by overlaying the labeled antibody or fragment onto the sample. This procedure allows for the determination of the presence, absence, amount and location of a polypeptide of interest.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibodies herein. Bioluminescence is a type of chemiluminescence found in biological systems in which a catalytic protein increases the efficiency of the chemiluminescent reaction. The presence of a bioluminescent protein is determined by detecting the presence of luminescence. Preferred bioluminescent compounds for purposes of labeling antibodies are luciferin, luciferase and aequorin.

In one embodiment, the presence (or absence) of a polypeptide associated with liver related disease in a sample (e.g., a cell, cell lysate, tissue, whether in vivo or in vitro) can be established by contacting the sample with an antibody and then detecting a binding complex. The presence of a polypeptide associated with resistance to liver related disease is a diagnostic and prognostic of resistance to liver related disease or more particularly cirrhosis. The presence of a polypeptide associated with susceptibility to liver related disease is a prognosis and diagnosis of susceptibility to liver related disease, liver related disease, or cirrhosis.

In another embodiment, the level of expression or composition of a polypeptide associated with liver related disease in a test sample is compared with the level of expression of the same polypeptide in a control sample. A control sample can be a known level of expression of the polypeptide, or a level of expression in a sample from a healthy individual or from a different organ from the tested individual.

Alterations in the level of expression or composition of a liver related disease polypeptide may be indicative of susceptibility or resistance to liver related disease. In one example, a test sample from an individual is assessed for a change in expression (e.g., level of transcription) and/or composition (e.g., splicing variants) of a polypeptide associated with susceptibility to liver related disease. Detection of an increased level of expression of a polypeptide associated with susceptibility to liver related disease may be a prognosis or diagnosis of, for example, an onset of liver related disease or an increased susceptibility to related disease. On the contrary, detection of a reduced level of a polypeptide associated with susceptibility to related disease may be indicative of, for example, a reduced susceptibility to liver related disease or an effective treatment against liver related disease. Detection of an increased level of a polypeptide associated with resistance to liver related disease may be a prognosis or diagnosis of, for example, increased immunity to liver related disease or an effective treatment regimen against liver related disease. On the other hand, detection of a reduced level of a polypeptide associated with resistance to liver related disease may be a prognosis or diagnosis of, for example, decreased immunity to liver related disease or an ineffective treatment regimen against liver related disease. Similarly, detection of an increase in compositions (e.g., derivatives, variants, splicing variants) associated with susceptibility to liver related disease is a prognosis and diagnosis of an onset or more severe symptoms of liver related disease or cirrhosis, while detection of an increase in compositions associated with resistance to liver related disease is a prognosis and diagnosis for immunity or reduced risk for developing liver related disease, or cirrhosis.

Kits useful in diagnosis and prognosis include reagents comprising, for example, nucleic acid probes or primers (for amplification, reverse transcriptase and detection), restriction enzymes (e.g., for RFLP analysis), allele-specific probes, antisense nucleic acids, antibodies and other protein binding probes any of which may be labeled.

V. Screening Assays and Agents

The following assays may be used to identify agents that modulate the expression of the nucleic acids and polypeptides associated with liver related disease. Such agents may, for example, interact with regulatory sequences of liver related disease polypeptides, interact with mRNA transcript of liver related disease polypeptides, interact with post-translated liver related disease polypeptides or interact with molecules that bind liver related disease polypeptides (“binding molecule”) to result in an alteration in liver related disease polypeptide expression and/or activity.

Examples of agents include: transcription factors, binding molecules, antisense nucleic acids, PNAs, mimetics, small or large organic or inorganic molecules, polypeptides (e.g., soluble peptides, or Ig-tailed fusion peptides), antibodies (monoclonal, polyclonal, humanized, anti-idiotypic, chimeric or single chain antibodies, Fab, F(ab′)₂, Fab expression library fragments, and epitope-binding fragments thereof), fusion proteins, prodrugs and any fragments, derivatives, variants or complements of any of the above. Such agents can be used separately or in combination.

Agents identified via these assays may be utilized to prevent, treat, diagnose and prognosticate liver related disease especially cirrhosis. For example, whereby liver related disease results from an overall lower level of polypeptides associated with resistance to liver related disease, agents that enhance or stimulate the expression or activity of such polypeptides may be used to treat or prevent liver related disease. In another example, whereby liver related disease results from the upregulation of polypeptides associated with susceptibility to liver related disease, agents that inhibit or diminish the expression or activity of such polypeptide may be used to treat or prevent liver related disease.

1. Screening Assays for Agents that Enhance/Inhibit Polypeptide Expression by Interacting with Coding Nucleic Acids

In one embodiment, agents that modulate (enhance or inhibit) the level of expression of a liver related disease polypeptide can be identified by comparing the level of expression of such polypeptide in the presence of a test agent and in a control. A control can be in the absence of the test agent or a previously established level of expression. A solution or sample containing nucleic acids encoding a liver related disease polypeptide can be contacted with a test agent. A solution can comprise, for example, of cells or cell lysates containing the liver related disease gene as well as other elements necessary for transcription/translation. Cells not suspended in solution as well as animal models may also be used.

If the level of expression of the liver related disease polypeptide is greater by an amount that is statistically significant from the level of expression in the control, then the test agent is an agonist of liver related disease gene expression or activity. If the level of expression in the presence of the test agent is less by an amount that is statistically significant from the level of expression in the control, then the test agent is an antagonist of the expression of associated gene. The level of expression polypeptides can be evaluated, for example, by determining the level of mRNA and/or any other method herein or known in the art, including but not limited to Northern analysis, Western blotting and antibodies.

Using a similar method, agents that modulate the expression of associated gene variants associated with resistance or with susceptibility to liver related disease can be identified. Preferably an agent is an agonist to the expression of associated genomic region variants associated with resistance to liver related disease or an antagonist to the expression of associated genomic region variants associated with susceptibility to liver related disease. More preferably, an agent is both an agonist to the expression of associated genomic region variants associated with resistance to liver related disease and an antagonist of associated genomic region variants associated with susceptibility to liver related disease.

2. Screening Assays for Agents that Enhance/Inhibit Polypeptide Expression by Interacting with Regulatory Regions

In another embodiment, agents that modulate liver related disease polypeptides by interacting with a liver related disease regulatory region (e.g., introns, 5′ and 3′ untranslated regions and uORF's) are provided. For example, agents that modulate transcription or translation of nucleic acids herein (e.g., transcription factors) can be identified by contacting a solution containing non-coding nucleic acids associated with liver related disease operably linked to a reporter gene with a test agent. After contact with the test agent, the level of expression of the reporter gene (e.g., the level of mRNA or polypeptide) is assessed and compared with the level of expression in a control (e.g., the level of expression in the absence of a test agent or a level of expression that has previously been established). If the level of expression in the test sample is greater than the level of expression in the level of expression in the control sample by a statistically significant amount, then the test agent is an agonist of expression. If the level of expression in the test sample is less than the level of expression in a control sample by a statistically significant amount, then the test agent is an antagonist of the expression.

In a preferred embodiment, an agent is an antagonist to the expression of associated genomic region variants associated with susceptibility to liver related disease. In another preferred embodiment, an agent is an agonist to the expression of associated genomic region variants associated with resistance to liver related disease. Preferably, an agent is both an antagonist to the expression of liver related disease variants associated with susceptibility to liver related disease and an agonist to the expression of liver related disease variants associated with resistance to liver related disease.

3. Screening Assays for Agents that Enhance/Inhibit Polypeptide Activity

In another embodiment, agents that alter (enhance or inhibit) the activity of polypeptides associated with liver related disease (e.g., enhance the presence of certain splicing variants or enhance binding activity) are identified by contacting a test agent with a cell, cell lysate or a solution containing nucleic acids and/or polypeptides associated with liver related disease and comparing that activity of the polypeptides with their activity in a control (in absence of the test agent or a previously established level activity). If the level of activity of polypeptides associated with liver related disease is enhanced by an amount that is statistically significant from the level of activity of the same polypeptides in a control, then the agent is an agonist of such polypeptides. If the level of activity of polypeptides associated with liver related disease is less than the level of activity in a control by an amount that is statistically significant, then the agent is an antagonist to the activity of polypeptides associated with resistance to liver related disease.

In a preferred embodiment, an agent is an agonist of the activity of polypeptides associated with resistance to liver related disease. In another preferred embodiment, an agent is an antagonist of the activity of polypeptides associated with susceptibility to liver related disease. Preferably, an agent is both an agonist of the activity of polypeptides associated with resistance to liver related disease and an antagonist of the activity of polypeptides associated with susceptibility to liver related disease.

4. Protein Agents that Bind Liver Related Disease Polypeptides

In another embodiment, assays can be used to identify protein agents that interact or bind one or more of the polypeptides herein, e.g., a liver related disease polypeptide.

In one embodiment, a yeast two-hybrid system, such as that described by Fields and Song (Fields, S. and Song, O., (1989) Nature 340:245-246), can be used to identify polypeptides that interact with one or more liver related disease variants. A yeast two-hybrid system employs two vectors. The first vector has a DNA binding domain; the second, a transcription activation domain. Each domain is fused to a sequence encoding a different polypeptide. If the polypeptides interact with one another, transcriptional activation can be achieved, and transcription of specific markers can be used to identify the presence of interaction and transcriptional activation. In one example, a first vector contains a nucleic acid encoding a DNA binding domain and a liver related disease polypeptide, and a second vector contains a nucleic acid encoding a transcription activation domain and test polypeptide which may potentially interact with the liver related disease polypeptide (e.g., a binding agent). Incubation of yeast containing the first vector and the second vector under appropriate conditions (e.g., mating conditions such as those used in the Matchmaker system from Clontech) allows for the identification of colonies that express the markers of interest. These colonies can be examined to identify the polypeptide(s) that interact with the liver related disease polypeptide tested. The binding molecules may be use as agents, which alter the activity of expression of a liver related disease polypeptide as described above.

In another embodiment, a protein microchip may be used to identify polypeptides that bind to liver related disease polypeptides or any other polypeptide herein. A protein microchip or microarray is provided having one or more protein complexes and/or antibodies selectively immunoreactive with a polypeptide of interest. Protein microarrays are becoming increasingly important in both proteomics research and protein-based detection and diagnosis of diseases. The protein microarrays in accordance with this embodiment are be useful in a variety of applications including, e.g., large-scale or high-throughput screening for compounds capable of binding to the protein complexes or modulating the interactions between the interacting protein members in the protein complexes.

Protein microarrays can be prepared in a number of methods known in the art. An example of a suitable method is that disclosed in MacBeath and Schreiber, (2002) Science, 289:1760-1763. Essentially, glass microscope slides are treated with an aldehyde-containing silane reagent (SuperAldehyde Substrates purchased from TeleChem International, Cupertino, Calif.). Nanoliter volumes of protein samples in a phosphate-buffered saline with 40% glycerol are then spotted onto the treated slides using a high-precision contact-printing robot. After incubation, the slides are immersed in a bovine serum albumin (BSA)-containing buffer to quench the unreacted aldehydes and to form a BSA layer that functions to prevent non-specific protein binding in subsequent applications of the microchip. Alternatively, as disclosed in MacBeath and Schreiber, proteins or protein complexes of the present invention can be attached to a BSA-NHS slide by covalent linkages. BSA-NHS slides are fabricated by first attaching a molecular layer of BSA to the surface of glass slides and then activating the BSA with N,N′-disuccinimidyl carbonate. As a result, the amino groups of the lysine, aspartate, and glutamate residues on the BSA are activated and can form covalent urea or amide linkages with protein samples spotted on the slides. See MacBeath and Schreiber, Science, 289:1760-1763 (2000).

Another example of a useful method for preparing a protein microchip is disclosed in PCT Publication Nos. WO 00/4389A2 and WO 00/04382. First, a substrate or chip base is covered with one or more layers of thin organic film to eliminate any surface defects, insulate proteins from the base materials, and to ensure uniform protein array. Next, a plurality of protein-capturing agents (e.g., antibodies, peptides, etc.) are arrayed and attached to the base that is covered with the thin film. Proteins or protein complexes can then be bound to the capturing agents forming a protein microarray. The protein microchips are kept in flow chambers with an aqueous solution.

The protein microarrays herein can also be made by the method disclosed in PCT Publication No. WO 99/36576, which is incorporated herein by reference. For example, a three-dimensional hydrophilic polymer matrix, i.e., a gel, is first dispensed on a solid substrate such as a glass slide. The polymer matrix gel is capable of expanding or contracting and contains a coupling reagent that reacts with amine groups. Thus, proteins and protein complexes can be contacted with the matrix gel in an expanded aqueous and porous state to allow reactions between the amine groups on the protein or protein complexes with the coupling reagents thus immobilizing the proteins and protein complexes on the substrate. Thereafter, the gel is contracted to embed the attached proteins and protein complexes in the matrix gel.

The protein microchips of the present invention can also be prepared with other methods known in the art, e.g., those disclosed in U.S. Pat. Nos. 6,087,102, 6,139,831, 6,087,103; PCT Publication Nos. WO 99/60156, WO 99/39210, WO 00/54046, WO 00/53625, WO 99/51773, WO 99/35289, WO 97/42507, WO 01/01142, WO 00/63694, WO 00/61806, WO 99/61148, WO 99/40434, all of which are incorporated herein by reference.

5. Agents that Interfere with Liver Related Disease Interaction with Binding Agents

The polypeptides herein can interact in vivo with one or more cellular or extracellular binding agents (e.g., polypeptides, nucleic acids, etc.) to form a complex. Agents that disrupt such interaction can be used to regulate the activity of the liver related disease polypeptides herein. Assays that assess the impact of a test agent on the activity of a liver related disease polypeptide in relation to a cellular or extracellular binding agent are provided. These assays involve the preparation of a reaction mixture containing a liver related disease polypeptide and a cellular or extracellular binding agent and for a time sufficient to allow the two products to interact and bind thus forming a complex.

To test an agent for inhibitory activity, reaction mixtures are prepared in the presence and absence of the test agent. The test agent can be initially included in the reaction mixture or added at a time subsequent to the addition of the liver related disease polypeptide and its cellular or extracellular binding agent. Control reaction mixtures can be incubated without the test agent or with a placebo. Formation of complexes between liver related disease polypeptides and cellular or extracellular binding agents are detected both in the control and test reaction mixtures. The formation of a complex in the control reaction but not in the reaction mixture containing the test agent indicates that the compound interferes with the interaction of the liver related disease polypeptide and the cellular or extracellular binding agent. Additionally, complex formation within the reaction mixtures containing the test agent and liver related disease polypeptide can also be compared to complex formation in a reaction mixture containing the test agent and a variant liver related disease polypeptide. This comparison can be important in those cases in which it is desirable to identify agents that disrupt interaction of a particular variant liver related disease polypeptide.

In either example, test agents that interfere with the interaction between the liver related disease polypeptides and the cellular or extracellular binding agents can be tested for interference, for example, by competition by adding the test agent to the reaction mixture prior to, post, or simultaneously within the liver related disease polypeptide and cellular or extracellular binding agents and assessing the difference in complex formation. Alternatively, test agents that disrupt formed complexes, (e.g., compounds with higher binding constants that displace one of the components from the complex) can be tested by adding the test agent to the reaction mixture after the complexes have been formed.

The ability or effectiveness of a test agent to bind to a liver related disease polypeptide or a cellular or extracellular binding agent can be assessed, for example, by coupling a test agent with a radioisotope or enzymatic label such that binding of the test agent to the liver related disease polypeptide can be determined by detecting the labeled with ¹²⁵I, ³⁵S, ¹⁴C, or ³H, either directly or indirectly, and the radioisotope detected by direct counting of radioemission or by scintillation counting. Alternatively, test agents can be enzymatically labeled with, for example, horseradish peroxidase, alkaline phosphatase or luciferase and the enzymatic label can be detected by determination of conversion of an appropriate substrate to a product.

In another embodiment, the ability of a test agent to interact with a liver related disease polypeptide can be assessed without the labeling of any of the interactants. For example, a microphysiometer can be used to detect the interaction of a test agent with a liver related disease polypeptide or a binding agent without the labeling of either the test agent, the liver related disease polypeptide or the binding agent. See McConnell, H. M. et al. (1992) Science 257:1906-1912. As used herein, a “microphysiometer” (e.g., CytosensorTu) is an analytical instrument that measures the rate at which a cell acidifies its environment using a light-addressable potentiometric sensor (LAPS). Changes in this acidification rate can be used as an indicator of the interaction between the binding agent and the liver related disease polypeptide.

6. Screening for Small Molecules

Agents that enhance or inhibit the expression and/or activity of liver related disease polypeptides can be obtained using any of the numerous approaches in combinatorial library methods known in the art, including: biological libraries; natural products libraries; spatially addressable parallel solid phase or solution phase libraries; synthetic library methods requiring deconvolution; the 'one-bead one-compound'library method; and synthetic library methods using affinity chromatography selection. The biological library approach is largely limited to polypeptide libraries, while the other four approaches are applicable to polypeptide, non-peptide oligomer or small molecule libraries of compounds. See Lam, K. S. (1997) Anticancer Drug Des. 12:145.

Non-peptide agents or small molecules are generally preferred because they are more readily absorbed after oral administration and have fewer potential antigenic determinants. Small molecules are also more likely to cross the blood brain barrier than larger protein-based pharmaceuticals. Methods for screening small molecule libraries for candidate protein-binding molecules are well known in the art and may be employed to identify molecules that bind to one or more of the liver related disease polypeptides herein. Briefly, liver related disease polypeptides may be immobilized on a substrate and a solution including the small molecules is contacted with the liver related disease polypeptide under conditions that are permissive for binding. The substrate is then washed with a solution that substantially reflects physiological conditions to remove unbound or weakly bound small molecules. A second wash may then elute those compounds that are bound strongly to the immobilized polypeptide. Alternatively, the small molecules can be immobilized and a solution of liver related disease polypeptides can be contacted with the column, filter or other substrate. The ability of a liver related disease polypeptide to bind to a small molecule may be determined by labeling (e.g., radio-labeling or chemiluminescence).

In another embodiment, electronic molecular modeling applies automatic algorithm to screen small molecule databases for ligands and molecules that interact or bind with liver related disease polypeptides or those in pathways therewith. See Meng et al., (1992) J. Comp. Chem. 15:505. In one example the DOCK3.5 is used to screen for small molecules that interact with liver related disease polypeptides, preferably the binding pocket of a liver related disease polypeptide. A “negative image” of the binding pocket on a protein surface is created. The image is created by the computational equivalent of placing atom-sized spheres into the binding pocket. A representative set of spheres are identified by DOCK3.5 that fit extremely well into the binding pocket. The generated spheres constitute an irregular grid that is matched to the atomic centers of potential ligands. The list of atom centers, or more conveniently the matrix of interatomic distances linking these atom centers forms a useful description of the binding site. The matrix of interatomic distances for the putative ligand is also made. The best mutual overlap of the two matrices is sought. This alignment specifies the orientation of the ligand relative to the negative image of the protein and thus docks the ligand into the protein's binding pocket.

Non-peptide agents or small molecule libraries can be prepared by a synthetic approach, but recent advances in biosynthetic methods using enzymes may enable one to prepare chemical libraries that are otherwise difficult to synthesize chemically. Small molecule libraries can also be obtained from various commercial entities, for example, SPECS and BioSPEC B.V. (Rijswijk, the Netherlands), Chembridge Corporation (San Diego, Calif.), Comgenex USA Inc., (Princeton, N.J.), Maybridge Chemical Ltd. (Cornwall, U.K.), and Asinex (Moscow, Russia). These small molecule libraries can be screening in a high throughput manner to identify one or more agents. For example, a high throughput screening assay for small molecules that was disclosed in Stockwell, B. R. et al., Chem. & Bio., (1999) 6:71-83, is a miniaturized cell-based assay for monitoring biosynthetic processes such as DNA synthesis and post-translational processes.

7. Immobilization Assays

In any embodiment herein, it may be desirable to immobilize either the liver related disease polypeptides, the test agent or other components of the assay (e.g., binding agents) on a substrate in order to facilitate the separation of bound polypeptides from unbound polypeptides, as well as to accommodate automation of the assay. A substrate can be any vessel suitable for containing the reactants. Examples of substrates include: microtiter plates, test tubes, and micro-centrifuge tubes. In one example, agents that bind a polypeptide of interest can be detected by anchoring either the polypeptide of interest (e.g., any polypeptide herein) or the test agent (e.g., antibody) to a substrate (e.g., microtiter plates) and then detecting complexes of the polypeptide of interest and test agent anchored to the substrate at the end of the reaction. Where the polypeptide of interest is anchored and the test agent is not anchored, the test agent can be labeled, either directly or indirectly.

In a preferred embodiment, microtiter plates are used as the solid phase, and the anchored component can be immobilized by non-covalent or covalent attachments. Non-covalent attachments can be achieved by simply coating the solid surface with a solution of the protein and drying. In another preferred embodiment, an immobilized antibody (preferably a monoclonal antibody) specific for the polypeptide to be immobilized can be used to anchor the polypeptide to the solid surface. The surface can be prepared in advance and stored.

In another embodiment, a fusion protein (e.g., a glutathione-S-transferase fusion protein) can be provided which adds a domain that allows the polypeptides, binding agents or test agents to be bound to a matrix or other solid support. A non-immobilized component is then added to the coated surface containing the anchored component. After the reaction is complete, unreacted components are removed (e.g., by washing) and complexes anchored on the solid surface are detected. Where the previously immobilized component is pre-labeled, the detection of label immobilized on the surface indicates that the complexes were formed. Where previously immobilized component is not pre-labeled, an indirect label can be used to detect complexes anchored on the surface, such as by using a labeled antibody specific for the immobilized component. The antibody can then be labeled or indirectly labeled with an anti-Ig antibody.

Alternatively, this reaction can be conducted in a liquid phase, the reaction products separated from unreacted components, and complexes are detected using, for example, an immobilized antibody specific for a polypeptide of interest or test agent to anchor the complexes formed in solution and a labeled antibody specific for the other component of the possible complex to detect anchored complexes.

In another embodiment, an assay performed in liquid phase has the preformed complexes of the liver related disease polypeptides and the cellular or extracellular binding agents prepared such that either the polypeptide or the binding agents are labeled, but the signal generated from the label is eliminated or diminished due to complex formation. The addition of a test agent that competes with and displaces one of the species from the performed complex will result in the generation of a signal above background.

In one particular embodiment, the liver related disease polypeptide is prepared using recombinant DNA techniques described herein and is fused to a glutathione-S-transferase (GST) gene using a fusion factor such as pGEX-5X-1, such that its binding activity is maintained in the resulting fusion product. The cellular or extracellular product thereof, is purified and used to raise a monoclonal antibody, using methods routinely practiced in the art. This antibody can be labeled with the radioactive isotope ¹²⁵I, for example by methods known in the art. In a substrate binding assay, the GST-liver related disease polypeptide fusion product is anchored, for example, to glutathione-agarose beads. The cellular or extracellular binding agents are then added in the presence or absence of the test agent in a manner that allows interaction and binding to occur. At the end of the reaction period, unbound material is washed away, and the labeled monoclonal antibody can be added to the system and allowed to bind to the complexed components. The interaction between the liver related disease polypeptide and the cellular or extracellular binding agents is detected by measuring the amount of radioactivity that remains associated with the beads. A successful inhibition of the interaction by the test agent will result in a decrease in measured radioactivity.

Alternatively, the GST bound liver related disease polypeptide fusion product and the interactive cellular or extracellular binding agent can be mixed together in liquid in the absence of the solid glutathione-agarose beads. The test agent is added either during or after the binding agent is allowed to interact with the GST-fusion polypeptide. This mixture is then added to the glutathione-agarose beads and unbound material is washed away. The extent of inhibition of the binding agent interaction can be detected by adding the labeled antibody and measuring the radioactivity associated with the beads.

The same techniques can also be employed using polypeptide fragments, derivatives, or variants that correspond to the binding domains of either the liver related disease polypeptides (e.g., BH3) or the cellular or extracellular binding agents, or both. Binding sites can be identified and isolated using any one of a number of methods known in the art, including for example site directed mutagenesis.

Alternatively, a liver related disease polypeptide can be anchored to a solid substrate using methods disclosed herein and allowed to interact with and bind its labeled binding agent, which has been previously treated with a proteolytic enzyme (e.g., trypsin). After washing, a short-labeled peptide comprising the binding domain (e.g., BH3) remains associated with the solid material, which can be isolated and identified by amino acid sequencing. Also, once the gene coding for the cellular or extracellular binding agent is obtained, short gene segment can be engineered to express binding fragments, which can then be tested for binding activity, purified and/or synthesized.

8. Agents that Enhance/Inhibit Genes in the Liver Related Disease Pathways

Liver related disease may further be prevented or treated by administering to a patient an agent that enhances or inhibits the expression or activity of genes in the associated gene pathways. Genes in the associated gene pathways are those that are upstream of the associated genomic regions whose gene products interact with, bind to, compete with, induce, enhance, or inhibit, directly or indirectly, the activity or expression of genes in the associated genomic regions, or any gene whose gene products are downstream of associated genomic regions, wherein the associated genomic region induces, enhances or inhibits the expression of activity of such gene products, directly or indirectly. Genes in the pathways of TRHDE, DRD3 and STAT1 are of particular relevance herein.

9. Potential Agents and Binding Sites

Agents that modulate the expression or activity of liver related disease polypeptides include: nucleic acids, antisense nucleic acids, polypeptides, fusion proteins, antibodies, binding molecules, prodrugs and small and large organic or inorganic molecules.

Any of the agents herein can also serve as “lead agents” in the design and development of new pharmaceuticals. For example, sequential modification of small molecules (e.g., amino acid residue replacement with peptides, functional group replacement with peptide or non-peptide compounds) is a standard approach in the pharmaceutical industry for the development of new pharmaceuticals. Such development generally proceeds from a lead agent, which is shown to have at least some of the activity of the desired pharmaceutical. In particular, when one or more agents having at least some activity of interest are identified, structural comparison of the molecules can greatly inform the skilled practitioner by suggesting portions of the lead agents that should be conserved and portions that may be varied in the design of new candidate compounds. This embodiment also encompasses means of identifying lead agents that may be sequentially modified to produce new candidate agents for use in the treatment of liver related disease. These new agents may be tested for therapeutic efficacy (e.g., in the cell-based or animal models described herein). This procedure may be iterated until compounds having the desired therapeutic activity and/or efficacy are identified.

10. Cell Based Assays and Animal Models

The agents herein can be tested for their ability to prevent, ameliorate or treat symptoms associated with liver related disease, especially cirrhosis, using cell-based system assays, animal models and/or clinical trials. Cell-based systems can be useful for identifying agents that ameliorate symptoms associated with liver related disease. Such symptoms include jaundice, kidney failure, liver failure, hepatitis A, fatty liver and inflammation of the liver. Cell-based systems include cells that express one or more of the liver related disease polypeptides herein and exhibit cellular phenotypes associated with resistance or susceptibility to liver related disease. Cell-based systems include recombinant transgenic cell lines derived from animals containing one or more cells expressing one or more of the nucleic acids herein. Preferably, such cells provide a continuous cell line. Cell-based systems also include non-recombinant cell lines preferably from primary tissues of patients having liver related disease or resistance to liver related disease.

A cell-based system having a phenotype of liver related disease can be exposed to an agent suspected of ameliorating phenotypic states associated with susceptibility to liver related disease at a sufficient concentration and for a time sufficient to elicit such an amelioration response in the exposed cells. After exposure, the cells can be examined to determine whether the phenotypic states have been altered such that the phenotype has been eliminated and the cells resemble normal phenotypes or phenotypes of resistance to liver related disease.

Animal models can be used to determine toxicity, efficacy and/or mechanism of action of the agents identified herein. Animal models for liver related disease include both non-recombinant and recombinant transgenic animals. Non-recombinant animal models for liver related disease include, for example, dog and murine models. Murine models can be created, for example, by administering to an animal an effective amount of alcohol or a drug to elicit a response or symptom associated with liver related disease. Such animal models can then be exposed to an agent suspected of ameliorating liver related disease.

Additionally, recombinant animal models exhibiting phenotypic states of liver related disease or resistance thereto can be engineered, for example, by introducing nucleic acids associated with susceptibility or resistance, respectively. Techniques for making a transgenic animal are known in the art. Such techniques include, for example, pronuclear microinjection disclosed in U.S. Pat. No. 4,873,191; retrovirus mediated gene transfer into germ-lines disclosed in Van der Putten et al., (1985) Proc. Natl. Acad. Sci. USA, 82:6148-6152; gene targeting in embryonic stem cells disclosed in Thomson et al., (1989) Cell 56:313-321; electroporation of embryos disclosed in Lo, (1983) Mol. Cell. Biol. (3) 1803-1814; and sperm-mediated gene transfer disclosed in Lavitrano et al, (1989) Cell 57:717-723. Nucleic acids can also be introduced into some, but not all cells of an animal to create a mosaic animal. Selective introduction into and activation of a particular cell type is discussed, for example, in Lasko et al. (1992) Proc. Natl. Acad. Sci. USA 89:6232-6236. An engineered sequence includes preferably at least part of the target nucleic acid sequence. This disrupts the endogenous target sequence upon integration of the engineered target gene sequence into the animal's genome.

In a preferred embodiment, the nucleic acids herein are used to over-express polypeptides associated with resistance to liver related disease. In another preferred embodiment, the nucleic acids herein are used to underexpress polypeptides associated with susceptibility to liver related disease. To overexpress a polypeptide, for example, a nucleic acid encoding the polypeptide of interest can be ligated to a regulatory sequence that can drive the expression of the polypeptide in the animal cell type of interest. Such regulatory regions are well known to those skilled in the art. In another example, a non-genic nucleic acid (e.g., an intron or a regulatory sequence) may be introduced alone to drive the production of a polypeptide of interest. To underexpress an endogenous polypeptide, a nucleic acid encoding a transcription factor that down-regulates the polypeptide or a nucleic acid that produces a variant or inactive polypeptide may be introduced into the genome of an animal such that the endogenous expression will be inactivated. In addition to, or in the alternative, a non-genic nucleic acid herein (e.g., an intron nucleic acid) may be introduced separately to override native regulatory region.

Any of the animal models disclosed herein can be used to identify agents capable of ameliorating, treating or preventing symptoms associated with susceptibility to liver related disease. For example, animal models can be exposed to a compound suspected of exhibiting an ability to ameliorate one or more symptoms associated with liver related disease at a sufficient concentration and for a time sufficient to elicit an ameliorating response in the exposed animal. The response of the exposed animal can be monitored by assessing change in symptoms. Any treatments that diminish symptom associated with liver related disease or susceptibility thereto should be considered as a candidate for human therapy. Dosages of test agents can be determined by deriving dose-response curves.

VI. Pharmaceutical Compositions

Any of the agents and compositions identified herein may be produced in quantities sufficient for pharmaceutical administration and/or testing. 1

Pharmaceutical compositions can be formulated in accordance with the routine procedures adapted for administration to human beings. Often, pharmaceutical compositions are formulated with an acceptable carrier or excipient. See Remington's Pharmaceutical Sciences, Gennaro, A., (ed., Mack Publishing Co. 1990).

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof.

Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The pharmaceutical compositions can include, if desired, auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The pharmaceutical compositions, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides.

The pharmaceutical compositions and their physiologically acceptable salts and solvates can be formulated for administration by inhalation or insufflation (either through the mouth or the nose, or oral, buccal, parenteral, or rectal administration). For administration by inhalation, the compositions are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

For oral administration, the pharmaceutical compositions can take the form of tablets or capsules prepared by conventional means with pharmaceutically acceptable excipients such as binding agents, fillers, disintegrants, or wetting agents, sweeteners, including, pregelatinised maize starch, polyvinylpyrrolidone, hydroxypropyl methylcellulose, fillers, lactose, microcrystalline cellulose, calcium hydrogen phosphate, lubricants, magnesium stearate, talc, silica, potato starch or sodium starch glycolate, sodium lauryl sulphate. mannitol, lactose, starch, magnesium stearate, polyvinyl pyrollidone, sodium saccharine, cellulose and magnesium carbonate. The tablets can be coated by methods well known in the art. Preparations for oral administration can be suitably formulated to give controlled release of the active compound.

Liquid preparations for oral administration can take the form of solutions, syrups, or suspensions, or they can be presented as a dry product for constitution with water or other suitable vehicle before use. Such liquid preparations can be prepared by conventional means with pharmaceutically acceptable additives such as suspending agents, e.g., sorbitol syrup, cellulose derivatives, or hydrogenated edible fats; emulsifying agents, e.g., lecithin or acacia; non-aqueous vehicles, e.g., almond oil, oily esters, ethyl alcohol, or fractionated vegetable oils; and preservatives, e.g., methyl or propyl-p-hydroxybenzoates or sorbic acid. The preparations can also contain buffer salts, flavoring, coloring, and/or sweetening agents as appropriate.

In particular, the liquid preparations can be administered in a beverage. Such beverage can be alcoholic, non-alcoholic beverage or a health beverage. Such beverage may comprise one or more of the agents or compositions herein as well as, optionally, any one or more of the following: alcohol fructose, vitamins, electrolyte substitutes, caffeine, amino acids, minerals, artificial and natural sweeteners, milk or dry-milk powder and other additives and preserving agents.

Examples of vitamins that may be included are components of the vitamin B complex, such as vitamin B1, B2, B6, B12, biotin, niacin, pantothenic acid, folic acid, adenine, choline, adenosine phosphate, orotic acid, pangamic acid, camitine, 4-aminobenzoic acid, myo-inositol, liponic acid and/or amygdaline. In the body, vitamin B1, also known as thiamin, is converted into thiamin-pyrophosphate, a coenzyme in a number of reactions in which C—C bonds are cleaved. It can also be added as thiamin hydrochloride. Vitamin B2, also known as riboflavin, is reabsorbed in the small intestines, converted into FMN (flavin mononucleotide) and, in the liver, into FAD (flavin-adenine-dinucleotide), both of which are coenzymes in redox reactions, e.g. with alcohol dehydrogenase. Vitamin B6, also known as pyridoxal, pyrodoxin and pyridoxamine, is a component of pyridoxal-5-phosphate, which is a cofactor in glycogen degradation and in amino acid metabolism, e.g. as a coenzyme of decarboxylases. Preferably, this substance is admixed into the beverage in the form of pyridoxin hydrochloride. Vitamin B12, also known as cyanocobalamine, has a complex structure and is a component of cobalamine-coenzymes, with methyl-cobalamine and cobalamide, e.g., being involved in rearrangements with hydrogen migration. Biotin, also known as vitamin B7, is covalently bound to carboxylases. Niacin, also known as B3, is a generic name for nicotinic acid and nicotinamide. Niacin is a component of NAD and its phosphate, NADP, and is one of the most important hydrogen transmitters in the cell having a protective and anabolic effect on the body. Pantothenic acid, also known vitamin B3 or B5, has a precursor function for coenzyme A which assumes a central position in metabolism. Folic acid, or vitamin B9, is a component of the coenzyme tetrahydrofolate. Vitamin C may further be probided.

Preferably, the beverage composition comprises components of the vitamin B complex in the following parts by weight, based on a total of 15,000-20,000 parts by weight of the dry substance: vitamin B1, 0.1-10 parts by weight, preferably 1 part by weight; vitamin B2, 0.1-10 parts by weight, preferably 1.5 parts by weight; vitamin B6, 0.1-10 parts by weight, preferably 1.5 parts by weight; biotin, 0.01-1 parts by weight, preferably 0.1 parts by weight; niacin, 0.1-100 parts by weight, preferably 10-30 parts by weight; pantothenic acid, 0.1-100 parts by weight, preferably 1-10 parts by weight; vitamin B12, 0.0001-0.1 parts by weight, preferably 0.001-0.01 parts by weight; folic acid, 0.01-10 parts by weight, preferably 0.1 parts by weight, and/or vitamin C, 0.1-500 parts by weight, preferably 50 parts by weight.

It is advantageous for the beverage to comprise of amino acids, in particular L-glutamine and/or L-arginine. Amino acids play an important role in the various metabolic processes of the human body and may have positive effect on the alcohol degradation of the body. In particular, L-glutamine and L-arginine promote alcohol degradation, and may be admixed in the beverage according to the following parts by weight, based on a total of 15,000-20,000 parts by weight of dry substance: L-arginine, 20-2,000 parts by weight, preferably 200 parts by weight; and/or L-glutamin, 10-1,000 parts by weight, preferably 100 parts by weight.

Caffeine is optionally added at 0.1-100 parts by weight, preferably 25 parts by weight, based on a total of 15,000-20,000 parts by weight.

Examples of mineral that may be used include magnesium, potassium, zinc and calcium. In particular, potassium and magnesium play an important role in metabolism and are involved in many ATP-catalyzed enzyme reactions, and zinc is a component of alcohol-dehydrogenase, which involved in the metabolism of alcohol. Mineral may be added separately, in combination, and/or in combination with other food additives, e.g. as magnesium glycerophosphate, potassium citrate (acid regulator), zinc gluconate (fruit acid) and calcium pantothenate. Minerals are preferably added at the following parts by weights, based on a total of 15,000-20,000 parts by weight of the dry substance: magnesium, 10-1,000 parts by weight, preferably 100 parts by weight; potassium 10-1,000 parts by weight, preferably 100 parts by weight; zinc, 0.1-100 parts by weight, preferably 10 parts by weight; calcium 10-1,000 parts by weight, preferably 100 parts by weight.

A tastier beverage may further include sugars and/or artificial sweeteners. Both artificial and natural sweeteners may be added to sweeten the compositions herein. Besides fructose, any other sugar may be admixed, such as glucose, galactose, lactose, etc. Artificial sweeteners include, for example, aspartame, saccharine and cyclamate as well as any other commercially available artificial sweeteners.

Furthermore, the compositions herein may comprise of further additives, in particular flavoring agents, preserving agents, coloring agents, antioxidants, electrolytes, enzymes, plant extracts, glycerolphosphates, acid regulators and/or acidifiers, in particular fruit acids.

A beverage may be carbonated or non-carbonated, and may be combined or based on liquids such as fruit juices, milk, tea, coffee, water etc. Moreover, alcohol may be admixed to the beverage herein.

The compositions can be formulated for intravenous administration. Compositions used for intravenous administration are typically solutions in sterile isotonic aqueous buffer. Where necessary, the compositions may also include a solubilizing agent and a local anesthetic to ease pain at the site of the injection. Generally, the ingredients are supplied either separately or mixed together in unit dosage format, for example, as a dry lyophilized powder or water free concentrate in a hermetically sealed container such as an ampule indicating the quantity of active agent. Where the composition is to be administered by infusion, it can be dispensed with an infusion bottle containing sterile pharmaceutical grade water, saline or dextrose/water. Where the compositions are administered by injection, an ampule of sterile water for injection or saline can be provided so that the ingredients may be mixed prior to administration.

The compositions can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions, or emulsions in oily or aqueous vehicles, and can contain formulatory agents such as suspending, stabilizing, and/or dispersing agents. Alternatively, the active ingredient can be in powder form for constitution with a suitable vehicle, e.g., sterile pyrogen-free water, before use.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carver material, is packaged in a squeeze bottle or in admixture with a pressurized volatile, normally gaseous propellant, e.g., pressurized air.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example, subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration. Pharmaceutical packs or kits comprising one or more containers filled with one or more of the ingredients of the pharmaceutical compositions disclosed herein are also provided. Optionally, associated with such containers can be a notice in the form prescribed by a governmental agency regulating the manufacture, use or sale of pharmaceuticals or biological products, which notice reflects approval by the agency of manufacture, use of sale for human administration. The packs or kits can be labeled with information regarding mode of administration, sequence of drug administration (e.g., separately, sequentially or concurrently) or the like. The packs or kits may also include means for reminding the patient to take the therapy. The packs or kits can comprise of a single unit dosage of the combination therapy or a plurality of unit dosages. In particular, the compositions can be separated, mixed together or present in a single vial or tablet. Compositions assembled in a blister pack or other dispensing means are preferred. Unit dosages provided are preferably dependent on the pharmacodynamics of each agent and administered in FDA approved dosages in standard time courses.

VIII. Methods for Treatment

The agents and pharmaceutical compositions herein can be used as prophylactic or therapeutic treatment of liver related disease, in particular cirrhosis. Liver related disease may result from excessive levels of certain gene products (e.g., liver related disease polypeptides) or deficient levels of other gene products (e.g., polypeptides associated with resistance to liver related disease).

1. Indications for Treatment

Preferable indications for treatment involve scarring of liver tissue or liver dysfunction, especially those associated with liver related disease. Other indications for treatment include, but are not limited to the following: edema, ascites, bruising, jaundice, loss of blood clotting abilities, toxins in the blood and/or brain, portal hypertension, kidney failure, immune system dysfunction, hepatitis, copper build-up in organs, varices, hypersensitivity to medications, loss of appetite, nausea, weakness, loss of weight, fatigue, exhaustion.

Other indications associated with liver related disease include: acute liver failure, biliary atresia, cholestatic liver disease, cystic disease of the liver, fatty liver, galactosemia, gallstones, Gilbert's syndrome, hemochromatosis, porphyria, primary biliary cirrhosis, primary sclerosing cholangitis, Reye's syndrome, sarcoidosis, Wilson's disease, arthritis, rheumatoid arthritis, allergic rhinitis, asthma, cardiovascular disease, chronic obstructive pulmonary disease, inflammatory bowel syndrome, and multiple sclerosis.

2. Methods for Administration

The agents and pharmaceutical compositions herein can be administered separately or in combination, in an amount effective to treat an indication of interest. For example, a patient diagnosed with or afflicted by a liver related disease, especially cirrhosis, may be administered a therapeutically effective amount of an inhibitor of polypeptides associated with susceptibility to liver related disease to reduce the level of activity and/or expression of such polypeptides. In the alternative, a patient diagnosed with or afflicted by a liver related disease, especially cirrhosis, may be administered a therapeutically effective amount of an agonist of polypeptides associated with resistance to liver related disease to reduce the level of activity and/or expression of such polypeptides. More preferably, a patient diagnosed with or afflicted by a liver related disease, especially cirrhosis, is administered a combination treatment of both inhibitors of polypeptides associated with susceptibility to liver related disease and agonists of polypeptides associated with resistance to liver related disease. Such combination treatment may require lower dosages due to the synergetic effect of both compounds.

The agents and pharmaceutical compositions may be administered or co-administered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds and/or compositions may also be administered or co-administered in slow release dosage forms. Other suitable methods include gene therapy using rechargeable or biodegradable devices, particle acceleration devices (“gene guns”) and slow release polymeric devices. The pharmaceutical compositions herein can also be administered as part of a combinatorial therapy with other agents.

The combination of therapeutic agents and compositions may be administered by a variety of routes, and may be administered or co-administered in any conventional dosage form. Co-administration in the context of this invention is defined to mean the administration of more than one therapeutic in the course of a coordinated treatment to achieve an improved clinical outcome. Such co-administration may also be coextensive, that is, occurring during overlapping periods of time. For example, a associated genomic region antisense may be administered to a patient before, concomitantly, or after the administration of an inhibitor of liver related disease polypeptides.

In a preferred embodiment, a pharmaceutical compound is administered orally, and more preferably is self-administered. For example, a beverage comprising one or more agents or pharmaceutical compositions may be administered to prevent, ameliorate or treat liver related disease. Such beverage may be administered prior to-, concomitantly with and/or post alcohol consumption. The dosage of active ingredients may be based on the composition, its interaction with other compounds, or more preferably the amount of alcohol consumed by a patient.

3. Gene Replacement Therapy

In another embodiment, nucleic acids can be introduced into recipient cells using techniques such as gene replacement therapy.

Preferably, one or more nucleic acids associated with resistance to liver related disease may be inserted into appropriate cells within a patient, using vectors such as adenovirus, adeno-associated virus and retrovirus vectors. Nucleic acids can also be introduced into cells via particles, such as liposomes. Other techniques for direct administration involve stereotactic delivery of such sequences to the site of the cells in which the sequences are to be expressed.

Methods for introducing nucleic acids into mammalian cells are well known in the art. Generally, the nucleic acid is directly administered in vivo into a target cell or a transgenic mouse that expresses SP-10 promoter operably linked to a reporter gene. This can be accomplished by any methods known in the art, e.g., by constructing it as part of an appropriate nucleic acid expression vector and administering it so that it becomes intracellular, e.g., by infection using a defective or attenuated retroviral or other viral vector (U.S. Pat. No. 4,980,286), by direct injection of naked DNA, by use of microparticle bombardment (e.g., a gene gun; Biolistic, Dupont), by coating with lipids or cell-surface receptors or transfecting agents, by encapsulation in liposomes, microparticles, or microcapsules, by administering it in linkage to a peptide which is known to enter the nucleus, or by administering it in linkage to a ligand subject to receptor-mediated endocytosis (Wu and Wu, (1987) J. Biol. Chem. 262:4429-4432), which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992; WO 92/22635 dated Dec. 23, 1992; W092/20316 dated Nov. 26, 1992; W093/14188 dated Jul. 22, 1993; WO 93/20221 dated Oct. 14, 1993).

Additional methods which may be utilized to increase the overall level of expression of a nucleic acid include using targeted homologous recombination methods to modify the expression characteristics of an endogenous sequence in a cell or microorganism by inserting a heterologous DNA regulatory element such that the inserted regulatory element is operatively linked with the endogenous sequence in question. Targeted homologous recombination can thus be used to activate transcription of an endogenous nucleic acid which is transcriptionally silent, (e.g., not normally expressed or expressed at very low levels), or to enhance the expression of an endogenous sequence which is normally expressed.

Further, the overall level of expression of polypeptides associated with resistance to liver related disease may be increased by the introduction of cells that express such polypeptides associated with resistance to liver related disease, preferably autologous cells, into a patient at positions and in numbers which are sufficient to prevent or ameliorate symptoms or conditions associated with liver related disease. Such cells may be either recombinant or non-recombinant. In a preferred embodiment, such cells are healthy liver cells.

When the cells to be administered are non-autologous cells, they can be administered using well-known techniques that prevent a host immune response against the introduced cells from developing. For example, the cells may be introduced in an encapsulated form that, while allowing for an exchange of components with the immediate extracellular environment, does not allow the introduced cells to be recognized by the host immune system.

The amounts of therapeutic agents or compositions to be administered can vary, according to determinations made by one of skill, but preferably are in amounts effective to create reduce inflammation and/or reverse cirrhosis at a desired site. Treatment compositions and dosages can be specifically tailored to each situation based on an individual patient's pharmacogenomics (response to a drug), phenotype, genotype and the compositions used for treatment. Preferably, for co-administration, the total amounts are less than the total amounts for each pharmaceutical compound added together. For the slow-release dosage form, appropriate release times can vary, but preferably should last from about 1 hour to about 6 months, most preferably from about 1 week to about 4 weeks. Formulations for slow release dosage can vary as determinable by one of skill, according to the particular situation and as generally taught herein.

The LD₅₀ (the lethal dose to 50% of the population) and the ED₅₀ (the effective dose in 50% of the population) of a pharmaceutical composition can be determined using cell cultures or animal models following standard pharmaceutical procedures. The dose ratio of lethal and effective doses is the therapeutic index and is expressed as the ratio LD₅₀/ED₅₀ Compounds that exhibit large therapeutic indices are preferred. Compounds that exhibit toxic side effects can also be used, but care should be taken to design a delivery system that targets such compounds to the site of affected tissue to minimize potential damage to uninfected cells.

When using cell culture to estimate the therapeutically effective dose, the dosage of such compounds lies preferably within a range of circulating concentrations that include the ED₅₀ with little or no toxicity. A dose can also be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography.

The combination of therapeutic agents may be used in the form of kits. The arrangement and construction of such kits is conventionally known to one of skill in the art. Such kits may include containers for containing the inventive combination of therapeutic agents and/or compositions, and/or other apparatus for administering the inventive combination of therapeutic agents and/or compositions.

The invention will be further described by the following non-limiting examples. The teachings of all publications cited herein are incorporated herein by reference in their entirety.

### EXAMPLES

### Example 1

The entire human genome was scanned to identify common variants (and others) using microarray technology platforms such as described in U.S. Ser. No. 10/106,097, entitled “Methods for Genomic Analysis”, filed on Mar. 26, 2002, assigned to the same assignee as the present application; U.S. Ser. No. 10/284,444, entitled “Chromosome 21 SNPs, SNP Groups and SNP Patterns,” filed on Oct. 31, 2002, assigned to the same assignee as the present application; and 10/042,819, entitled “Whole Genome Scanning,” filed on Jan. 7, 2002, assigned to the same assignee as the present application, all of which are incorporated herein by reference. The microarrays are manufactured using a process adapted from semiconductor manufacturing to achieve cost effectiveness and high quality and were manufactured by Perlegen Sciences, Inc.

### Example 2

Variants identified were grouped into haplotype blocks using methods disclosed in U.S. Ser. No. 10/106,097, entitled “Methods for Genomic Analysis”, filed Mar. 26, 2002 (Attorney Docket #1005U-1), incorporated herein by reference. Representative variants and haplotype blocks from an entire human chromosome (chromosome 21) are disclosed in, for example, Patil, N. et al, “Blocks of Limited Haplotype Diversity Revealed by High-Resolution Scanning of Human Chromosome 21” Science 294, 1719-1723 (2001) and the associated supplemental materials, incorporated herein by reference.

### Example 3

Individuals from populations in Mexico that drink excessively (at least 80 grams of alcohol per day for men and 40 grams of alcohol per day for women, for at least 5 years) were selected for an association study. All individuals were free of hepatitis B and hepatitis C virus. Individuals clinically diagnosed with cirrhosis were identified as cases. Individuals not clinically diagnosed with any of the following symptoms were identified as controls: cirrhosis, jaundice, spider angiomas, palmar erythema, abdominal collateral circulation, ascitis, hepatic encephalopathy, esophageal varices, portal hypertension and esophageal varices. Some diagnoses were verified using one or more of the following biochemical criteria: abnormal aminotransferases, glutamil transpeptidase, alkaline phosphatase, decreased serum albumin, increased serum globulin, decreased prothrombin concentration. Some diagnoses were further verified using imaging techniques such as ultrasonography or CT scanning. The association studies were preformed on 454 cases and 505 controls.

### Example 4

Two blood samples (a “primary” and a “back-up”) were collected from each individual. The samples ranged between 2-10 milliliters each. The DNA from each sample was purified using commercially available products, for example, Roche “DNA Isolation Kit for Mammalian Blood” and the amount of DNA present was measured using optical density analysis. The yield from a single blood sample was between 2 and 300 micrograms of genomic DNA. Over 90% of samples yielded greater than 100 micrograms of DNA, and 95% of samples yielded greater than 40 micrograms of genomic DNA. The concentrations of each DNA sample were adjusted to create stock solutions with DNA concentrations between 0.4 μg/μl and 0.6 μg/μl.

To further evaluate the purified DNA, 0.1 microgram of DNA was analyzed by agarose gel electrophoresis on a 0.8% agarose gel containing 3-5 μl of 10 mg/ml ethidium bromide per 100 ml of agarose. Two μl of the DNA stock solution were added to enough water to create a 0.05 μg/μl dilution. Standard loading buffer was added to the sample and the sample was loaded onto the gel. The gel was run at 150 volts for 40-45 minutes, and then subjected to ultraviolet light so that a photograph could be taken. A strong band of genomic DNA on the gel was an indication that the majority of the DNA was not degraded; a smear on the gel was an indication that the DNA was largely degraded and possibly not useful for further testing. Those that were largely degraded were not used for further testing. Polymerase chain reaction (PCR) was used to assess the quality of the DNA as a template for amplification. The post-PCR DNA was analyzed by agarose gel electrophoresis on a 0.8% agarose gel containing 1 μg/ml of ethidium bromide. A strong band of amplified DNA on the gel was an indication that the DNA was of a high enough quality to be used in amplification reactions; the lack of such a band was an indication that the DNA was not useful for further testing. It was found that the presence of a large band of genomic DNA on the gel containing the purified pre-PCR DNA was a good predictor of success in the subsequent amplification reaction. Hence, for some samples, the subsequent PCR assessment was omitted.

### Example 5

The back-up samples were stored at −80° C., while the primary samples were subjected to a “normalization” procedure to equilibrate the DNA concentrations of each sample. After normalization, the samples were also tested for population stratification so that a correction could be applied to get an equal population structure value for each pooled sample. Stratification and correction assays are further described in U.S. provisional patent application Ser. No. Unassigned (Attorney Docket No. 1047P-1), entitled “Stratification Assay.” Equal volumes from each case sample were pooled together to form a “case pool;” and equal volumes of each control sample were pooled to form a “control pool.” Remaining portions of case or control samples were stored at −80° C.

### Example 6

The case pool and control pool were each separated into two equal pools for a total of four pools, (e.g., two identical case pools and two identical control pools). Each pool was separately subjected to PCR using primers designed to amplify genomic DNA containing single nucleotide polymorphisms (SNPs). The PCRs were performed in 384-well plates containing primer pairs to which PCR reaction cocktail, DNA template (one of the pools discussed supra), a Taq antibody (and its buffer), and a long-range DNA Polymerase were added. The final DNA concentration in the PCR was 100 ng/μl. The PCR plates were sealed prior to PCR. Long-range PCR was performed for approximately 13.5 hours. The thermocycler block was allowed to reach 90° C. before the PCR plates were placed in the thermocycler. The thermocycler program used for the PCR is identified in Table 2:

TABLE 2

| Step | Action |
|---|---|
| 1 | Incubate at 95° C. for 3 min |
| 2 | Incubate at 94° C. for 2 seconds |
| 3 | Incubate at 64° C. for 15 minutes |
| 4 | goto [step] “2” (for 10 subsequent cycles) |
| 5 | Incubate at 94° C. for 2 seconds |
| 6 | Incubate at 64° C. for 15 minutes* |
| 7 | goto [step] “5” (for 28 subsequent cycles) |
| 8 | Incubate at 62° C. for 60 minutes |
| 9 | Hold at 4° C. |

*increased by 20 seconds for each subsequent cycle

### Example 7

The post-PCR pools were purified using commercially available centrifugal filter device. Using a spectrophotometer, the concentration of each post-PCR pool was measured twice, once for a 1:200 fold dilution and once for a 1:300 fold dilution. These two measurements were then averaged to get a final concentration. Then, each pool was diluted to achieve a final DNA concentration of approximately 1.5 μg/μl. If the concentration of the pool was between 1.3 μg/μl and 1.7 μl, the pool was considered to be close enough to 1.5 μg/μl and the concentration was not adjusted. If the pool had a concentration above 1.7 μg/μl, then enough molecular grade water was added to lower the concentration to 1.5 μg/μl. If the pool had a concentration of less than 1.3 μg/μl, then it was concentrated to raise the concentration to 1.5 μg/μl using a commercially available concentrating centrifugal filter device. Finally, the concentration of each ˜1.5 μg/μl pool was rechecked using a spectrophotometer.

To check the quality of the post-PCR pools, aliquots of each were subjected to agarose gel electrophoresis in a 0.8% agarose gel containing 1 μg/ml ethidium bromide submerged in 1×TBE buffer. Aliquots containing approximately 3 μg of amplified DNA were added to loading buffer prior to being transferred to wells in the gel. Controls such as a commercially available DNA ladder and a known quantity of genomic DNA were also included on the gel. The gel was run at 250-275 volts for approximately 30 minutes and then photographed while illuminated by UV light. A post-PCR pool was deemed to be of good quality if the brightness of its band on the gel approximated that of the genomic DNA control.

### Example 8

Post PCR-pools were subjected to fragmentation by DNase I digestion. Each fragmentation reaction was performed in a 2 ml Eppendorf tube as follows. First, a buffered solution containing 0.0029 U/μl DNase I was added to 9.6 μg of post-PCR DNA in a total volume of 37 μl and placed at 37° C. for approximately eight minutes. Then the reaction was transferred to a 95° C. heat block for 10 minutes to denature the DNase I. After DNase I denaturation, the Eppendorf tube was placed on ice for at least five minutes and any condensation on the walls of the tube was spun down using a picofuge.

The success of each fragmentation reaction was examined by gel electrophoresis. Two microliters of each fragmentation reaction was added to 8 μl of gel-loading dye, and 5μl of the mixture was loaded onto an Invitrogen-Novex Precast gel (4-20% TBE gel) in 1×TBE buffer. A DNA ladder was also loaded onto the gel. Electrophoresis was performed at approximately 80 volts until the samples had migrated out of the wells (approximately five minutes), and the voltage was then increased to 132-146 volts for approximately 40 minutes. The gel was stained with 1×TBE containing 0.01% ethidium bromide for one minute at room temperature. Finally, the gel was photographed while being illuminated with UV light. For a fragmentation reaction to be deemed of good quality, the reaction appeared as a “smear” of fragments with the majority of the fragments between 40 and 100 base pairs in length. If the fragmentation reaction appeared to be of good quality, the next step was a labeling reaction as described below.

### Example 9

First, 1.5 μl of biotin mix stock (1 mM stock consisting of 0.5 mM of each of biotin 16-dUTP and biotin 16-ddUTP) was added to each tube containing a completed fragmentation reaction of good quality. Next, 1 μl (25 units) of native TdT (terminal transferase) (Boehringer Mannheim) or 1 μl (200 units) of recombinant TdT (Roche) was added to each tube. The fluid in the tubes was mixed and spun down in the picofuge prior to placement in a preheated thermocycler. The labeling reactions were incubated at 37° C. for 90 minutes, then at 95° C. for 10 minutes, and finally held at 4° C.

### Example 10

Each fragmented, labeled, post-PCR pool was separated into two pools for a total of four case pools and four control pools. Each of these eight pools was applied to a microarray containing oligonucleotides complementary to the genomic DNA that was amplified. Both strands of the amplified PCR product were probed for approximately 1.7 million variants across the genome using microarray oligonucleotide probes. Since there are generally two alleles for a given variant locus, the microarray contained both alleles of the complementary oligonucleotides at each variant position so that the amplified DNA could be screened for both alleles of a given variant simultaneously. A total of 228 different microarrays were used for each pool (control and case). Minor allele frequencies that varied significantly between the case group and control group were are characterized as being associated with related disease. Results were verified by genotyping individual samples for variants that were potentially associated with the case or control group based on the pooled analysis.

### Example 11

Prior to application to an microarray, 37.5 μl of a labeled, pooled sample, were combined with 187.5 μl of a hybridization solution comprising 130 μl 5M TMACl (tetramethylammonium chloride), 2.2 μl 1M Tris (pH 8), 2.2 μl 1% Triton X-100, 2.2 μl 5 nM control oligo b-948, 2.2 μl 10 mg/ml herring sperm DNA, and 48.7 μl H₂O. This mixture (225 μl total volume) was heated for 10 minutes at 95° C., spun down in a picofuge, and placed in a thermocycler where it was incubated at 95° C. for 10 minutes, then held at 50° C. Then, 200 μl of the pooled sample was transferred to a microarray that had been warmed at 50° C. The microarray containing the pooled sample is placed in a 50° C. hybridization oven where it is rotated at 25 rpm overnight (14 to 19 hours) such that the pooled sample is allowed to flow freely over the microarray during the incubation.

After incubation, the microarray was removed from the hybridization oven and the 200 μl sample was removed and stored at −20° C. Then, the microarray was washed with 200 μl of 1×MES/0.01% Triton X-100. The microarray was inverted several times to ensure that the wash solution moved freely over the surface of the microarray prior to removing the wash solution by vacuum suction.

Next, 200 μl of the “First Stain Solution” (174 μl of 1×MES/0.01% Triton X-100, 25 μl of 20 mg/ml of acetylated BSA, and 1 μl of 1 mg/ml streptavidin) was added to each microarray. The microarray was inverted several times to ensure that the First Stain Solution moved freely over the surface of the microarray. Then, the microarray was rotated at 25 r.p.m. for 15 minutes at room temperature. Next, the microarray was washed with 1×MES/0.01% Triton X-100 wash solution in a Perlegen PFS1200 Fluidics Station. When the wash was finished the microarray was removed from the fluidics station and the 1×MES/0.01% Triton X-100 wash solution was removed by vacuum suction.

Next, 200 μl of the “Second Stain Solution” (175 μl of 1×MES/0.01% Triton X-100, 25 μl of 20 mg/ml acetylated BSA, and 0.5 μl of 0.5 mg/ml biotinylated anti-streptavidin) was added to each microarray. The microarray was inverted several times to ensure that the Second Stain Solution moved freely over the surface of the microarray. Then, the microarray was rotated at 25 r.p.m. for 15 minutes at room temperature. Next, the microarray was washed with 1×MES/0.01% Triton X-100 wash solution in a PFS1200 Fluidics Station. When the wash was finished the microarray was removed from the fluidics station and the 1×MES/0.01% Triton X-100 wash solution was removed by vacuum suction.

Then, 200 μl of the “Third Stain Solution” (174 μl of 1×MES/0.01% Triton X-100, 25 μl of 20 mg/ml acetylated BSA, and 1 μl of 0.2 mg/ml streptavidin Cy-chrome) was added to each microarray. The microarray was inverted several times to ensure that the Third Stain Solution moved freely over the surface of the microarray. Then, the microarray was rotated at 25 r.p.m. for 15 minutes at room temperature. Next, the microarray was washed with 1×MES/0.01% Triton X-100 wash solution in a PFS1200 Fluidics Station. When the wash was finished the microarray was removed from the fluidics station and the 1×MES/0.01% Triton X-100 wash solution was removed by vacuum suction.

Then, a wash solution of 6×SSPE/0.01% Triton X-100 was added to the microarray. The microarray was inverted several times to ensure that the 6×SSPE/0.01% Triton X-100 moved freely over the surface of the microarray before it was removed by vacuum suction. Next, a wash solution of 0.2×SSPE/0.005% Triton X-100 that had been prewarmed to 37° C. was added to the microarray, which was then incubated at 37° C. for 30 minutes. The 0.2×SSPE/0.005% Triton X-100 was removed by vacuum suction and a solution of 1×MES/0.01% Triton X-100 was added to the microarray. The microarray was then inverted several times before the 1×MES/0.01% Triton X-100 was removed by vacuum suction. Finally, fresh 1×MES/0.01% Triton X-100 was added to the microarray prior to storage at 4° C.

### Example 12

On the same days the microarrays were stained and washed, they were scanned using an arc scanner. After scanning, the microarrays were removed from the scanner, wrapped in foil and stored at 4° C. The scan files generated by the scanner were then analyzed by software programs designed to interpret intensity data from microarrays. This software allowed discrimination of hybridization patterns that distinguished the case pools from the control pools. The data were analyzed according to the methods in U.S. Ser. No. Unassigned, filed on Ap. 3, 2003, entitled “Apparatus and Methods for Analyzing and Characterizing Nucleic Acid Sequences” assigned to the assignee of the present application. The nucleic acids listed in Table 1 were identified as strongly associated with the case or control group.

What is claimed is:

1. An isolated nucleic acid that specifically hybridizes to a genomic sequence from 10 kb upstream to 10 kb downstream of a liver related disease nucleic acid, for use in diagnostics, prognostics, prevention, treatment, or study of liver related disease, wherein said liver related disease nucleic acid is a gene containing a base at a position selected from the group of: base position 72677975 on chromosome 12, base position 31737404 on chromosome 15, base position 180740253 on chromosome 2, base position 65319504 on chromosome 18, base position 797949941 on chromosome 16, base position 112175641 on chromosome 4; base position 16152579 on chromosome 4, base position 115123242 on chromosome 3, base position 34165683 on chromosome 8, base position 191840092 on chromosome 2, base position 16343592 on chromosome 8, and base position 79942295 on chromosome 16.

2. A nucleic acid of claim 1 wherein the liver related disease is cirrhosis.

3. A nucleic acid of claim 1 wherein the nucleic acid specifically hybridizes to a reference sequence at a selected one of said base positions.

4. A nucleic acid of claim 1 wherein the nucleic acid specifically hybridizes to a variant from a reference sequence at a selected one of said base positions.

5. A nucleic acid of claim 1 wherein the nucleic acid specifically hybridizes to a variant in a common haplotype block with a selected one of said base positions.

6. A method for assaying the presence of a nucleic acid associated with resistance or susceptibility to liver related disease in a sample, comprising:
contacting said sample with a nucleic acid recited in claim 1 under stringent hybridization conditions; and
detecting a presence of a hybridization complex.

7. A method for diagnosing or prognosticating liver related disease comprising obtaining a sample from a patient; contacting the sample with a nucleic acid of claim 1; and detecting the presence or absence of a hybridization complex, wherein the presence or absence of a hybridization complex is a diagnostic of liver related disease.

8. An expression vector comprising an isolated nucleic acid from claim 1 operably linked to a reporter gene.

9. An expression vector comprising an isolated nucleic acid from claim 1 operatively linked to a regulatory sequence.

10. A recombinant host cell comprising the expression vector of claim 9.

11. A method for producing a transgenic knock-out mouse for use in the study of liver related disease, comprising the steps of:
disrupting one or more of the nucleic acids in any one of claim 1; and
introducing said disruption into the genomic DNA of said mouse by homologous recombination with a DNA targeting construct in an embryonic stem cell such that the targeting construct is stably integrated into the genome of said mouse.

12. A transgenic knock-out mouse of claim 11 for use in the study of liver related disease, wherein the disruption has been introduced into the mouse's genome by homologous recombination with a DNA targeting construct in an embryonic stem cell such that the targeting construct is stably integrated in the genome of said mouse.

13. An isolated polypeptide encoded by a nucleic acid of claim 1 for use in diagnostics, prognostics, prevention, treatment, or study of liver related disease.

14. An antibody, or an antigen-binding fragment thereof, which selectively binds to a polypeptide in claim 13 for use in diagnostics, prognostics, prevention, treatment, or study of liver related disease.

15. A fusion protein comprising an isolated polypeptide of claim 13 for use in diagnostics, prognostics, prevention, treatment, or study of liver related disease.

16. A method for assaying the presence or amount of a polypeptide of claim 14 for use in diagnostics, prognostics, prevention, treatment, or study of liver related disease, comprising:
contacting a sample with an antibody of claim 13 under conditions appropriate for binding;
assessing the sample for the presence or amount of binding of the antibody to the polypeptide.

17. A method for diagnosing liver related disease comprising comparing the level of expression or activity of a polypeptide in claim 13 in a test sample from a patient with the level of expression or activity of the same polypeptide in a control sample wherein a difference in the level of expression or activity between the test sample and control sample is indicative of liver related disease.

18. A method for identifying an agent that can alter the level of activity or expression of a polypeptide of claim 13 for use in diagnostics, prognostics, prevention, treatment, or study of liver related disease, comprising:
contacting a cell, cell lysate, or the polypeptide, with an agent to be tested;
assessing a level of activity or expression of the polypeptide of claim 13; and
comparing the level of activity or expression of the polypeptide with a control sample in an absence of the agent, wherein if the level of activity or expression of the polypeptide in the presence of the agent differs by an amount that is statistically significant from the level in the absence of the agent then the agent alters the activity or expression of the polypeptide.

19. An agent that alters the activity or expression identified by the method of claim 18.

20. An agent of claim 18 wherein the agent is selected from the group consisting of: a nucleic acid, an antisense nucleic acid, a ribozyme, a polypeptide, an antibody, a prodrug, a fusion protein, a mimetic, a binding molecule and a small molecule.

21. A method for identifying an agent for interaction of a polypeptide encoded by a nucleic acid of claim 1 comprising:
contacting the polypeptide, and the binding molecule with an agent to be tested;
assessing the interaction of the polypeptide with the binding molecule.

22. An agent which alters the interaction of a polypeptide encoded by a nucleic acid in claim 1 and a binding molecule identified according to the method of claim 21, selected from the group consisting of: a nucleic acid, an antisense nucleic acid, a ribozyme, a polypeptide, an antibody, a prodrug, a fusion protein, a mimetic, a binding molecule and a small molecule.

23. A method for identifying an agent which inhibits the expression or activity of protein encoded by a nucleic acid of claim 1 for use in diagnostics, prognostics, prevention, treatment, or study of liver related disease comprising:
contacting a cell, cell lysate, or a said polypeptide, with an agent to be tested;
assessing a level of activity or expression of said polypeptide, or fragment, derivative or variant thereof; and
comparing a level of activity or expression of the polypeptide, with a control sample in an absence of the agent; wherein if the level of activity or expression of the polypeptide, in the presence of the agent is reduced by an amount that is statistically significant from the level in the absence of the agent then the agent is an inhibitor.

24. An antagonist identified by the method in claim 23 wherein the antagonist is selected from the group consisting of: a nucleic acid, an antisense nucleic acid, a ribozyme, a polypeptide, an antibody, a prodrug, a fusion protein, a mimetic, a binding molecule and a small molecule.

25. A method for identifying an agent which enhances the expression or activity of a polypeptide encoded by a nucleic acid of claim 1 comprising:
contacting a cell, cell lysate, or the polypeptide with an agent to be tested;
assessing a level of activity or expression of the polypeptide; and
comparing the level of activity or expression of the polypeptide with the level of activity or expression in a control sample in an absence of the agent; wherein if the level of activity or expression of the polypeptide, in the presence of the agent is greater by an amount that is statistically significant from the level in the absence of the agent then the agent is an agonist.

26. An agonist identified in claim 25 wherein the agonist is selected from the group consisting of: a nucleic acid, an antisense nucleic acid, a ribozyme, a polypeptide, an antibody, a prodrug, a fusion protein, a mimetic, a binding molecule and a small molecule.

27. A method for identifying an agent which interacts with one or more polypeptides encoded by a nucleic acid of claim 1 for use in diagnostics, prevention, treatment, or study of liver related disease, wherein:
a first vector comprises a nucleic acid encoding a DNA binding domain and the polypeptide; and
a second vector comprises a nucleic acid encoding a transcription activation domain and a test agent,
wherein if the polypeptide of the first vector binds the test agent of the second vector, transcriptional activation is detected.

28. An agent identified by claim 27 for use in diagnostics, prevention, treatment, or study of liver related disease selected from the group consisting of: a polypeptide, an antibody, a prodrug, a fusion protein, a mimetic, a binding molecule and a small molecule.

29. A pharmaceutical composition for treatment of liver related disease in a mammal afflicted therewith comprising a therapeutically effective amount of an agent of any one of claims 13, 14, 19, 20, 22, 24, 26 or 28.

30. A method for treating or preventing liver related disease in a patient, comprising orally administering to the patient in need of such treatment an effective amount of an agent of any one of claim 29 or a pharmaceutically acceptable salt thereof.

31. A method of treating a mammalian patient for liver related disease, comprising adminstering to the patient a therapeutically effective amount of an antagonist or an agonist of pathway gene of a protein encoded by a nucleic acid recited in claim 1.

32. The method of claim 31 wherein the mammalian patient is a human.

33. The method of claim 31 wherein the liver related disease is cirrhosis.

34. The method of claim 31 wherein the antagonist is an antibody or an antisense therapeutic.

35. The method of claim 33 wherein the antagonist or the agonist is a small molecule therapeutic.

36. The method of claim 33 wherein the antagonist or the agonist is a polypeptide.

37. An isolated nucleic acid of 10-100 bases comprising at least 10 contiguous nucleotides, wherein the at least 10 contiguous nucleotides include or is immediately adjacent to a polymorphic site shown in Table 1.

38. The isolated nucleic acid of claim 37 comprising at least 20 contiguous nucleotides.

39. The isolated nucleic acid of claim 37 wherein the 3′ end of the at least 10 contiguous nucleotides is immediately adjacent to the polymorphic site.

40. An allele-specific oligonucleotide that specifically hybridizes to a segment of a nucleic acid shown in Table 1 including a polymorphic site.

41. An isolated nucleic acid comprising at least 10 contiguous nucleotides from a sequence shown in Table 1 including a polymorphic site, wherein the polymorphic site is occupied by a variant form shown in Table 1.

42. An isolated nucleic acid of claim 41 comprising at least 20 contiguous nucleotides from a sequence shown in Table 1 including a polymorphic site.

43. An expression vector comprising a nucleic acid comprising at least 10 contiguous nucleotides from a sequence shown in Table 1 including a polymorphic site, wherein the polymorphic site is occupied by a variant form shown in Table 1, operably linked to a promoter.

44. A host cell comprising an expression vector according to claim 43.

45. An isolated protein encoded by a gene, said gene containing a base identified in Table 1 which is not a reference allele.

46. An antibody that specifically binds to the protein as defined in claim 45 without specifically binding to a protein in which amino acid position is occupied by a reference amino acid encoded by a base specified in Table 1.

47. A method of making an animal model of liver disease, comprising modulating expression or activity of a protein in an animal, and exposing the animal to a condition that disposes the animal to develop a characteristic of liver related disease wherein the animal is a transgenic animal having a disrupted endogenous gene encoded by a nucleic acid recited in claim 1, whereby expression of a protein is inhibited or eliminated.

48. The method of claim 47 wherein the modulating of expression or activity comprises administering an siRNA that inhibits expression of said protein.

49. The method of claim 48 wherein the animal is a transgenic animal having a transgene comprising an exogenous gene operable linked to a regulatory sequence whereby expression of said protein is increased relative to a nontransgenic animal of the same species.

50. The method of claim 47 wherein the condition is exposure to alcohol.

51. An animal model of liver related disease, comprising an animal that has been genetically manipulated or treated with an agent to having increased or decreased expression or function of a protein recited in claim 13 relative to a control animal, whereby the modulated animal develops a characteristic of liver related disease.

52. A method of screening an agent for activity in treating liver related disease, comprising performing a primary screen to determine whether the agent affects level of expression or function of a protein recited in claim 13, and performing a secondary screen to determine whether the agent affects liver related disease in an animal.

53. The method of claim 52 wherein the primary screen measures binding of the agent to said protein.

54. The method of claim 52 wherein the primary screen measures capacity of the agent to agonize or antagonize said protein.

55. A method of screening an agent for activity in treating liver related disease, comprising exposing an animal model of liver related disease as defined in claim 53 to the agent; and determining whether the agent treats or inhibits further development of the disease in the animal model.

56. A method of screening an agent for activity in treating liver related disease, comprising exposing an animal in which expression of a protein recited in claim 13 is modulated to a condition that disposes the animal to develop a characteristic of liver related disease; exposing the animal to the agent; and determining whether the agent treats or inhibits development of the liver related disease.

57. A method of screening an agent for activity in treating liver related disease, comprising exposing an animal in which expression of a protein recited in claim 13 is modulated by said agent, and determining a response of the liver of the animal to the agent, the response indicating that the agent has activity in treating liver related disease.

58. A method of detecting presence or susceptibility to liver related disease in a patient, comprising determining a level of a protein recited in claim 13, and comparing the determined level of said protein to a baseline level of activity or function in a control patient, a difference in level indicating presence or susceptibility to liver related disease.

59. The method of claim 58 further comprising informing the patient or a relative thereof of presence or susceptibility to liver related disease.

60. The method of claim 58 further comprising performing a secondary test of liver related disease.

61. The method of claim 60 wherein the secondary test comprises determining the level of a liver specific enzyme.

62. The method of claim 61 wherein the secondary test comprises taking a liver biopsy.

63. The method of claim 58 further comprising administering a treatment regime effective to treat liver related disease.

64. A method of detecting presence or susceptibility to liver related disease in a patient, comprising determining whether the patient contains a variant form of protein recited in claim 13, the presence of the variable form indicating presence or susceptibility to liver related disease.

65. The method of claim 64 further comprising informing the patient or a relative thereof of presence or susceptibility to liver related disease.

66. The method of claim 65 further comprising performing a secondary test of liver related disease.

67. The method of claim 66 wherein the secondary test comprises determining the level of a liver specific enzyme.

68. The method of claim 66 wherein the secondary test comprises taking a liver biopsy.

69. The method of claim 64 further comprising administering a treatment regime effective to treat liver related disease.

70. A method of detecting presence or susceptibility to liver related disease in a patient, comprising determining whether the patient contains a polymorphic form of a protein recited in claim 13 and polymorphic forms in linkage disequilibrium with any of these, the presence of the polymorphic form indicating presence or susceptibility to liver related disease.

71. A method of inhibiting or treatment of liver related disease, comprising administering to a patient suffering from or at risk of liver related disease an agent that modulates expression or activity of a protein recited in claim 13 in a regime effective to inhibit or treat the liver related disease in the patient.

72. The method of claim 71 further comprising monitoring a property of the liver in the patient responsive to the administration.

73. The method of claim 71 further comprising counseling the patient to avoid conditions exacerbating liver related disease.

74. The method of claim 71 further comprising administering a second agent effective to inhibit or treat liver related disease.

75. The method of claim 71 wherein the mammalian patient is a human.

76. The method of claim 71 wherein the liver related disease is cirrhosis.

77. The method of claim 71 agent is an antagonist of a protein recited in claim 72.

78. The method of claim 77 wherein the antagonist is an antibody or an antisense molecule.

79. The method of claim 71 wherein the antagonist or the agonist is a small molecule.

80. The method of claim 71 wherein the antagonist or the agonist is a natural compound.

81. The method of claim 71 wherein the antagonist or the agonist is a polypeptide.

82. The method of claim 71 wherein the agent is selected from the group consisting of: a nucleic acid, an antisense nucleic acid, a ribozyme, a zinc finger protein, a polypeptide, an antibody, a prodrug, a fusion protein, a mimetic, a binding molecule and a small molecule.

83. A method for identifying a polymorphic site correlated with liver related disease or susceptibility thereto, comprising identifying a polymorphic site within a protein recited in claim 13 determining whether a variant polymorphic form occupying the site is associated with the disease or susceptibility thereto.

84. Use of an agent that modulates the expression or activity of a protein recited in claim 13 in the manufacture of a medicament of treatment or prophylaxis of liver related disease.

85. Use of an isolated nucleic acid that specifically hybridizes to a genomic sequence from 10 kb upstream to 10 kb downstream of a gene including one of the base positions recited in clam 1 for diagnosis, prognosis, prevention, treatment, or study of liver related disease.

86. A method of collecting samples for identifying genetic regions correlated with susceptibility to liver disease comprising collecting genomic samples from a first group of large alcohol consumers with cirrhosis and a second group of large alcohol consumers without cirrhosis wherein both of said first and second groups comprise individuals not clinically diagnosed with any of jaundice, spider angiomas, palmar erythema, abdominal collateral circulation, ascitis, hepatic encephalopathy, esophageal varices, portal hypertension, hepatitis, or esophageal varices.
